(19) United States
(12) Patent Application Publication
(10) Pub. No.: US 2020/0291394 A1
(43) Pub. Date: Sep. 17, 2020

(54) CONJUGATION OF PEPTIDES TO SPHERICAL NUCLEIC ACIDS (SNAS) USING TRACELESS LINKERS

(71) Applicant: Northwestern University, Evanston, IL (US)

(72) Inventors: Chad A. Mirkin, Wilmette, IL (US); Kacper Skakuj, Durham, NC (US); Shuya Wang, Evanston, IL (US)

(73) Assignee: Northwestern University, Evanston, IL (US)

(21) Appl. No.: 16/611,548

(22) PCT Filed: May 17, 2018
(86) PCT No.: PCT/US2018/033200

§ 371 Date: Nov. 7, 2019

Related U.S. Application Data
(60) Provisional application No. 62/507,591, filed on May 17, 2017.

(51) Int. Cl. C12N 15/11; A61K 39/00

(52) U.S. Cl. CPC C12N 15/11; A61K 39/0011; C12N 2310/532; A61K 2039/53; C12N 2310/3513; C12N 2310/3515; C12N 2310/17

(57) ABSTRACT

The present disclosure provides compositions and methods directed to combining spherical nucleic acid (SNA) components that are required for T-cell activation and proliferation.

### CROSS REFERENCE TO RELATED APPLICATIONS

This application claims the priority benefit under 35 U.S.C. § 119(e) of U.S. Provisional Patent Application No. 62/507,591, filed May 17, 2017, the disclosure of which is incorporated herein by reference in its entirety.

### STATEMENT OF GOVERNMENT SUPPORT

This invention was made with government support under U54 CA199091-01 awarded by the National Institutes of Health and N00014-15-1-0043 awarded by the Office of Naval Research. The government has certain rights in the invention.

### INCORPORATION BY REFERENCE OF MATERIALS SUBMITTED ELECTRONICALLY

This application contains, as a separate part of the disclosure, a Sequence Listing in computer readable form (Filename: 2017-092_Seqlisting.txt; Size: 1,458 bytes; Created: May 17, 2018), which is incorporated by reference in its entirety.

### BACKGROUND

Subtle changes in the chemical architecture of nanoparticle constructs can significantly influence biological function, including biodistribution properties,¹⁻³ drug release,⁴⁻⁶ and cellular internalization.⁷⁻¹⁰ To rationally design nanoparticles with desired properties, researchers should focus on characterizing the attributes which can be systematically changed and where structure-function relationships can begin to be defined. For example, SNA architectures, synthesized by arranging linear oligonucleotides on the surfaces of nanoparticle templates, have shown promise as probes in diagnostics¹¹ and as therapeutic lead compounds in medicine.¹² In the latter category, their ability to enter cells via endosomal pathways and agonize or antagonize toll-like receptors makes them highly promising immunomodulatory agents.¹³

### SUMMARY

In some aspects, the present disclosure provides chemical conjugation methods of peptides to nanoparticle vehicles for a targeted biological response.

Traceless linkers used to conjugate peptides to spherical nucleic acids (SNAs) can be used to maintain the unique properties of SNA architecture—for example and without limitation, efficient cellular uptake, and TLR activation—without sacrificing the biological efficacy of the delivered peptide. This property stems from the ability of the traceless linker to release the peptide in its native form, without irreversible chemical modifications, once inside the cell.

In some embodiments, the disclosure provides methods for the delivery of antigen peptide for immunostimulation targeting cancer cells. In further embodiments, the traceless linker conjugates a gp100 peptide antigen to an oligonucleotide. This traceless conjugate is then attached to an immunostimulatory SNA via DNA hybridization. When compared to other conjugation chemistries—non-cleavable, and non-traceless—the traceless linker affords superior immunostimulation, as measured by T-cell proliferation, while maintaining high levels of TLR-mediated APC activation. This effect is observed because only the traceless linker is able to release the antigen in its native form without chemical modifications after endocytosis.

The traceless conjugation strategy described in this disclosure can be applied to any SNA architecture that necessitates covalent conjugation of peptides to an SNA. These structures can be used to deliver biologically relevant peptides or proteins into cells by using the peptides as an SNA core, hybridizing them to the surface of the SNA, conjugating them to a different attachment moiety, or in any other manner that preserves the basic SNA architecture.

Advantages of the methods disclosed herein include but are not limited to the fact that the linkage does not require a cysteine to be present in the peptide sequence for traceless conjugation. The example provided herein demonstrates that the methods are not limited to using antigens that comprise cysteines. Further, the traceless nature prevents loss of biological activity of the peptide. In an example provided herein, the immune activation was improved by using a traceless linkage when compared to other linker chemistries.

In some aspects, the disclosure provides a spherical nucleic acid (SNA) comprising a nanoparticle and a double stranded oligonucleotide, wherein a first strand of the double stranded oligonucleotide comprises an associative moiety that allows association of the double-stranded oligonucleotide with the nanoparticle; a second strand of the double stranded oligonucleotide comprises an antigen that is attached to the second strand through a linker; wherein the first strand and the second strand comprise sequences that are sufficiently complementary to each other to hybridize to form the double stranded oligonucleotide. In some embodiments, first strand comprises an immunomodulatory nucleotide sequence. In further embodiments, the first strand comprises a sequence that is a toll-like receptor (TLR) agonist. In still further embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In some embodiments, the first strand comprises a CpG nucleotide sequence.

In some embodiments, the second strand comprises a carbamate alkylene dithiolate linker. In further embodiments, the second strand comprises Antigen-NH—C(O)—O—C₂₋₅alkylene-S—S—C₂₋₇alkylene-Oligonucleotide, or Antigen-NH—C(O)—O—CH₂—Ar—S—S—C₂₋₇alkylene-Oligonucleotide, and Ar comprises a meta- or para-substituted phenyl. In further embodiments, the second strand comprises Antigen-NH—C(O)—O—C₂₋₄alkylene-CH(X)—S—S—CH(Y)C₂₋₆alkylene-Oligonucleotide, and X and Y are each independently H, Me, Et, or iPr. In some embodiments, the second strand comprises Antigen-NH—C(O)—O—CH₂—Ar—S—S—CHXC₂₋₆alkylene-Oligonucleotide, and X is Me, Et, or iPr. In further embodiments, the second strand comprises an amide alkylene dithiolate linker. In some embodiments, the second strand comprise Antigen-NH—C(O)—C₂₋₅alkylene-S—S—C₂₋₇alkylene-Oligonucleotide. In further embodiments, the second strand comprises Antigen-NH—C(O)—CH(X)C₂₋₄alkylene-S—S—CH(Y)C₂₋₆alkylene-Oligonucleotide, and X and Y are each independently H, Me, Et, or iPr. In some embodiments, the second strand comprises a amide alkylene thio-succinimidyl linker. In still further embodiments, the second strand comprises Antigen-NH—C(O)—C₂₋₄alkylene-N-succinimidyl-S—C₂₋₆alkylene-Oligonucleotide.

In some embodiments, the antigen is a tumor associated antigen, a tumor specific antigen, a neo-antigen. In further embodiments, the antigen is OVA1, MSLN, P53, Ras, a melanoma related antigen, a HPV related antigen, a prostate cancer related antigen, an ovarian cancer related antigen, a breast cancer related antigen, a hepatocellular carcinoma related antigen, a bowel cancer related antigen, or human papillomavirus (HPV) E7 nuclear protein.

In further embodiments, the nanoparticle is a liposome. In some embodiments, the liposome comprises a lipid selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), and cholesterol.

In some embodiments, the associative moiety is tocopherol, cholesterol, 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-di-(9Z-octadecenoyl)-sn-glycero-3-phosphoethanolamine (DOPE), or 1,2-dihexadecanoyl-sn-glycero-3-phosphoethanolamine (DPPE).

In some embodiments, the double stranded oligonucleotide comprises RNA or DNA.

In some embodiments, a SNA of the disclosure further comprises an additional oligonucleotide. In further embodiments, the additional oligonucleotide comprises RNA or DNA. In some embodiments, the RNA is a non-coding RNA. In some embodiments, the non-coding RNA is an inhibitory RNA (RNAi). In still further embodiments, the RNAi is selected from the group consisting of a small inhibitory RNA (siRNA), a single-stranded RNA (ssRNA) that forms a triplex with double stranded DNA, and a ribozyme. In some embodiments, the RNA is a microRNA. In further embodiments, the DNA is antisense-DNA.

In some embodiments, the nanoparticle has a diameter of 50 nanometers or less.

In further embodiments, a SNA of the disclosure comprises about 10 to about 80 double stranded oligonucleotides. In some embodiments, a SNA of the disclosure comprises 75 double stranded oligonucleotides.

In some aspects, the disclosure provides a composition comprising a SNA of the disclosure in a pharmaceutically acceptable carrier. In some embodiments, the composition is capable of generating an immune response in an individual upon administration to the individual. In further embodiments, the immune response comprises antibody generation or a protective immune response.

In some aspects, the disclosure provides a vaccine comprising a composition of the disclosure, and an adjuvant. In some embodiments, the immune response is a neutralizing antibody response or a protective antibody response.

In some aspects, the disclosure provides a method of producing an immune response to cancer in an individual, comprising administering to the individual an effective amount of a composition or a vaccine of the disclosure, thereby producing an immune response to cancer in the individual.

In some aspects, the disclosure provides a method of inhibiting expression of a gene comprising hybridizing a polynucleotide encoding the gene with one or more oligonucleotides complementary to all or a portion of the polynucleotide, the oligonucleotide being an additional oligonucleotide as disclosed herein, wherein hybridizing between the polynucleotide and the oligonucleotide occurs over a length of the polynucleotide with a degree of complementarity sufficient to inhibit expression of the gene product. In some embodiments, expression of the gene product is inhibited in vivo. In further embodiments, expression of the gene product is inhibited in vitro.

In some aspects, the disclosure provides a method for up-regulating activity of a toll-like receptor (TLR) comprising contacting a cell having the TLR with a SNA of the disclosure. In some embodiments, the double stranded oligonucleotide comprises a TLR agonist. In further embodiments, the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13). In some embodiments, the method is performed in vitro. In further embodiments, the method is performed in vivo. In some embodiments, the cell is an antigen presenting cell (APC). In further embodiments, the APC is a dendritic cell. In some embodiments, the cell is a leukocyte. In still further embodiments, the leukocyte is a phagocyte, an innate lymphoid cell, a mast cell, an eosinophil, a basophil, a natural killer (NK) cell, a T cell, or a B cell. In some embodiments, the phagocyte is a macrophage, a neutrophil, or a dendritic cell.

In some aspects, the disclosure provides a method of immunizing an individual against cancer comprising administering to the individual an effective amount of a composition of the disclosure, thereby immunizing the individual against cancer. In some embodiments, the composition is a cancer vaccine. In further embodiments, the cancer is selected from the group consisting of bladder cancer, breast cancer, colon and rectal cancer, endometrial cancer, glioblastoma, kidney cancer, leukemia, liver cancer, lung cancer, melanoma, non-hodgkin lymphoma, osteocarcinoma, ovarian cancer, pancreatic cancer, prostate cancer, thyroid cancer, and human papilloma virus-induced cancer.

### BRIEF DESCRIPTION OF THE FIGURES

FIG. 1 shows (A) a schematic design of the immunostimulatory SNA. (B) Three distinct linker chemistries that were used to make the antigen-DNA conjugates 1-3: NDEC (traceless), SPDP (cleavable), and BMPS (non-cleavable), respectively. (C) Cholesterol-modified cyanine-5 (Cy5)-tagged anchor DNA, conjugate and anchor duplex, and SNA were characterized using 1% agarose gel, imaged by Cy5 fluorescence. (D,E) DLS shows an increase in diameter along with a decrease in zeta potential, measured at pH 7, between the bare liposome and the SNAs. Samples for DLS were prepared without the Cy5 modification.

FIG. 2 depicts antigen conjugation chemistry in immunostimulatory spherical nucleic acids (SNAs).

FIG. 3 depicts three linker types used to investigate the effect of antigen conjugation chemistry on T-Cell activation efficacy: non-cleavable (BMPS), cleavable (SPDP), and traceless (NDEC))(left panel). Each linker used delivers the antigen in with different degrees of chemical modification. The BMPS linker does not readily cleave, the SPDP linker cleaves via disulfide reduction but leaves a chemical pendant, while the NDEC linker also cleaves via disulfide reduction but regenerates the native peptide. Treatment of the conjugates with 10 mM glutathione, concentration representative of the intracellular environment, causes cleavage of labile linkers (PAGE gel)(right panel).

FIG. 4 shows the linker design, conjugate synthesis, DNA-Antigen conjugate structure, and degradation product for three linker designs.

FIG. 5 shows the kinetics of linker cleavage in the presence of 10 mM GSH. Both cleavable linker conjugates, NDEC and SPDP, showed an increase in fluorescence corresponding to a half-life of approximately 24 and 36 minutes, respectively.

FIG. 6 depicts examples of spherical nucleic acid synthesis and characterization, including changes in electrophoretic mobility, hydrodynamic radius, and zeta potential indicate formation of monodisperse SNAs. Compared to bare liposomes, the Z-average hydrodynamic diameter of particles increased by approximately 13 nm and the Zeta potential decreased by ˜22 mV. All the anchor strands are associated with the liposomal core, indicated by a lack of band corresponding to free anchor in the agarose gel.

FIG. 7 shows that no significant toxicity was observed by MTT assay using Dendritic cells with any of the three SNAs made with different linker conjugates.

FIG. 8 shows that SNAs deliver both adjuvant and antigen to dendritic cells. SNAs deliver both adjuvant CpG motif DNA (tagged with Cy5) and antigen gp100 peptide (tagged with AlexaFluor 488). The co-delivery efficiency is higher for adjuvant, antigen pairs formulated as an SNA compared to free in solution mixture. Confocal images show initial co-localization of antigen and adjuvant after delivery (2 h, R=0.70) but are directed to divergent trafficking pathways within four hours of treatment (4 h, R=0.33).

FIG. 9 shows (top panel) the delivery of Cy5-labled adjuvant (CpG) and AF488-labled antigen (gp100) is more efficient in an SNA form compared to a simple mixture of the two components. The bottom panel shows that the co-delivery efficiency of adjuvant and antigen are more efficient for SNAs compared to a simple mixture of the two components. This is representative data of FIG. 10.

FIG. 10 shows higher co-delivery of antigen and adjuvant in dendritic cells when they are structured in an SNA architecture compared to a simple mixture of the two components.

FIG. 11 shows that dendritic cell activation markers, CD40 and CD80, are upregulated compared to a media-only control. The upregulation was indistinguishable between all linker types. This indicated that the differences in linker chemistry do not significantly impact DC activation.

FIG. 12 shows that the potency of immunostimulatory SNAs, as measured by T-Cell proliferation, is affected by linker chemistry. Traceless linker (NDEC) provides a nearly eight-fold increase in potency as measured by EC₅₀ over the non-cleavable linker chemistry (BMPS), and a nearly three-fold increase over the cleavable but non-traceless counterpart (SPDP). Each measurement is an average of three technical replicates, standard deviations shown (left panel). A three parameter logistic dose-response curve was used to fit the data, 95% confidence bands of the fit are shaded. Set of chosen replicates of flow cytometry data at the 10 pM concentration is shown for the three linker types (right panel).

FIG. 13 shows the traceless linker that was used to conjugate CpG-complementary DNA to a prostate cancer antigen (TARP 2-9) and cleaves after incubation with reduced DTT.

FIG. 14 depicts ¹H NMR of 2-(2-Pyridinyldisulfanyl)ethanol. Solvent peaks indicated by asterisk: CHCl₃, CH₂Cl₂.

FIG. 15 depicts ¹H NMR of NDEC linker. Solvent peaks indicated by asterisk: CHCl₃, CH₂Cl₂, ethyl acetate, and water.

FIG. 16 depicts MALDI-TOF spectrum of peptide-DNA conjugates, collected with 2′,6′-dihydroxyacetophenone (DHAP) matrix in negative linear mode. Expected masses of conjugates are 7980 Da (BMPS conjugate), 7915 Da (SPDP conjugate), and 7931 (NDEC conjugate).

FIG. 17 shows results of an experiment in which the three gp100-DNA conjugates were treated with 10 mM glutathione (GSH) in 1×PBS (pH 7.4) for 2 hours at room temperature. Cleavable conjugates NDEC and SPDP showed a shift in electrophoretic mobility indicative of disulfide cleavage, while non-cleavable BMPS shows no change. Gel visualized with Sybr Gold DNA stain.

FIG. 18 shows MALDI-TOF spectra of conjugates before and after treatment with 10 mM glutathione in 2×PBS buffer (pH 7.4) for 24 hours at room temperature. Reactions were purified with C18 ZipTips before spotting on plate with α-cyano-4-hydroxycinnamic acid (CHCA) matrix, samples were collected in positive reflectron mode.

FIG. 19 depicts cleavage kinetics of the three conjugates were characterized using a fluorophore-quencher system.

FIG. 20 shows (A) Confocal microscopy images show gp100 antigen (AF488, green) and the CpG adjuvant (Cy5, red) inside mouse dendritic cells. (B,C) Flow cytometry measurements after a 15-minute incubation. Values are an average of three replicates (see FIG. 22 for additional replicates).

FIG. 21 shows MTT assay results for treatment with NDEC SNA.

FIG. 22 shows raw flow cytometry dot plots of adjuvant and antigen co-delivery in mouse dendritic cells. Q2 signifies cells showing co-delivery of both entities.

FIG. 23 depicts (A) Flow cytometry data showing CD8⁺ T-cell proliferation following incubation of pmel-1 splenocytes with the three types of SNAs at 10 pM concentration. (B) Dose-response curve of SNA treatment on T-cell proliferation. Average and standard deviation for three replicates are shown for each point (see FIG. 24 for additional replicates). The curves are three-parameter dose—response fits with a shaded 95% confidence interval of the fit. (C) Secreted cytokines quantified by ELISA, **** p<0.0001.

FIG. 24 shows raw flow cytometry data of T-cell proliferation using the eFluor 450 assay showing triplicate measurements for the three different SNA types at 10 pM and 1 pM concentrations by gp100 peptide.

FIG. 25 shows (A) Activation of mouse bone marrow derived DCs, using CD40 and CD80 markers, after treatment with different SNA structures at a 100 nM concentration or a medium only control. (B) Uptake of SNAs into mouse bone marrow derived dendritic cells by measuring MFI of Cy5-conjugated CpG under the same treatment conditions.

FIG. 26 shows results from experiments demonstrating that a carbamate linkage alone does not provide T-cell proliferation benefit. Shown are the various linkers utilized (left panel), T-cell proliferation data for each linker (middle panel), and EC50 data (right panel).

FIG. 27 shows additional linkers contemplated by the disclosure.

FIG. 28 demonstrates that dendritic cell surface markers show similar APC activation between linkers.

FIG. 29 depicts results of experiments showing that the presentation of OVA-I-MHC-I complex on the surface of dendritic cells varies between the linkers.

FIG. 30 depicts results of experiments showing that T-cell proliferation (dose-response curve, left panel) varied between the linker types. The right panel shows whole splenocytes incubated with SNAs at indicated concentrations for 72 hours.

FIG. 31 shows that additional steric bulk increased the rate of cyclization.

FIG. 32 shows results of experiments quantifying the rates of disulfide cleavage using the FITC-Eclipse quencher system.

### DETAILED DESCRIPTION

One of the properties that is possessed by SNAs is that they are potent sequence-specific stimulators of antigen presenting cells (APC). When loaded with peptide antigens, SNAs can be used to activate the immune system to train T-cells to specifically kill cancer cells. Herein, peptide chemical conjugation to an oligonucleotide, which is used to load SNAs with antigens via hybridization, is disclosed in the context of APC activation. In the case of cancer vaccines, the SNAs can also be used to carry antigens that provide selective training of the immune system through T-cell activation and proliferation. From a chemistry perspective, this created both a challenge and an opportunity. The present disclosure provides compositions and methods directed to combining SNA components that are required for T-cell activation and proliferation.

The way antigen molecules are incorporated in synthetic vaccines could impact not only quantities of antigen delivered to APCs but also the processing and chemical structure of the antigen. Indeed, for small molecule and peptide delivery, activity can be highly dependent on the type of conjugation chemistry employed.¹⁴⁻¹⁶ When designing the next generation of vaccines, such as immunostimulatory SNAs, it is imperative to understand the impact of the conjugation chemistry used to attach the antigen to the oligonucleotide that loads the antigen on the SNA construct. Specifically, since chemical modifications can influence peptide antigenicity, the present disclosure provides general strategies that can be used with a wide array of peptides, to deliver pristine antigens with no chemical appendages.

The terms “polynucleotide” and “oligonucleotide” are interchangeable as used herein.

The term “associative moiety” as used herein refers to an entity that facilitates the attachment of an oligonucleotide to a SNA.

An “immune response” is a response of a cell of the immune system, such as a B cell, T cell, or monocyte, to a stimulus, such as a pathogen or antigen (e.g., formulated as an antigenic composition or a vaccine). An immune response can be a B cell response, which results in the production of specific antibodies, such as antigen specific neutralizing antibodies. An immune response can also be a T cell response, such as a CD4+ response or a CD8⁺ response. B cell and T cell responses are aspects of a “cellular” immune response. An immune response can also be a “humoral” immune response, which is mediated by antibodies. In some cases, the response is specific for a particular antigen (that is, an “antigen-specific response”). An immune response can be measured, for example, by ELISA-neutralization assay. Exposure of a subject to an immunogenic stimulus, such as an antigen (e.g., formulated as an antigenic composition or vaccine), elicits a primary immune response specific for the stimulus, that is, the exposure “primes” the immune response.

As used in this specification and the appended claims, the singular forms “a,” “an,” and “the” include plural reference unless the context clearly dictates otherwise.

Spherical Nucleic Acids. Spherical nucleic acids (SNAs) comprise densely functionalized and highly oriented polynucleotides on the surface of a nanoparticle which can either be organic (e.g., a liposome) inorganic (e.g., gold, silver, or platinum) or hollow (e.g., silica-based). The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis(see, e.g., U.S. Patent Application Publication No. 2010/0233270, incorporated by reference herein in its entirety).

Nanoparticles are therefore provided which are functionalized to have a polynucleotide attached thereto. In general, nanoparticles contemplated include any compound or substance with a high loading capacity for a polynucleotide as described herein, including for example and without limitation, a metal, a semiconductor, a liposomal particle, insulator particle compositions, and a dendrimer (organic versus inorganic).

Thus, nanoparticles are contemplated which comprise a variety of inorganic materials including, but not limited to, metals, semi-conductor materials or ceramics as described in U.S. Patent Publication No 20030147966. For example, metal-based nanoparticles include those described herein. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles.

Liposomal particles, for example as disclosed in International Patent Application No. PCT/US2014/068429 (incorporated by reference herein in its entirety, particularly with respect to the discussion of liposomal particles) are also contemplated by the disclosure. Hollow particles, for example as described in U.S. Patent Publication Number 2012/0282186 (incorporated by reference herein in its entirety) are also contemplated herein. Liposomal particles of the disclosure have at least a substantially spherical geometry, an internal side and an external side, and comprise a lipid bilayer. The lipid bilayer comprises, in various embodiments, a lipid from the phosphocholine family of lipids or the phosphoethanolamine family of lipids. While not meant to be limiting, the first-lipid is chosen from group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, and a combination thereof.

In one embodiment, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO2, Sn, SnO2, Si, SO2, Fe, Fe+4, Ag, Cu, Ni, Al, steel, cobalt-chrome alloys, Cd, titanium alloys, AgI, AgBr, HgI2, PbS, PbSe, ZnTe, CdTe, In2S3, In2Se3, Cd3P2, Cd3As2, InAs, and GaAs. Methods of making ZnS, ZnO, TiO2, AgI, AgBr, HgI2, PbS, PbSe, ZnTe, CdTe, In2S3, In2Se3, Cd3P2, Cd3As2, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky, et al., J. Am. Chem. Soc., 112, 9438 (1990); Ushida et al., J. Phys. Chem., 95, 5382 (1992).

In practice, methods of increasing cellular uptake and inhibiting gene expression are provided using any suitable particle having oligonucleotides attached thereto that do not interfere with complex formation, i.e., hybridization to a target polynucleotide. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles particles, aggregate particles, isotropic (such as spherical particles) and anisotropic particles (such as non-spherical rods, tetrahedral, prisms) and core-shell particles such as the ones described in U.S. patent application Ser. No. 10/034,451, filed Dec. 28, 2002, and International Application No. PCT/US01/50825, filed Dec. 28, 2002, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers)

Suitable nanoparticles are also commercially available from, for example, Ted Pella, Inc. (gold), Amersham Corporation (gold) and Nanoprobes, Inc. (gold).

Also as described in US Patent Publication No. 20030147966, nanoparticles comprising materials described herein are available commercially or they can be produced from progressive nucleation in solution (e.g., by colloid reaction), or by various physical and chemical vapor deposition processes, such as sputter deposition. See, e.g., HaVashi, (1987) Vac. Sci. Technol. July/August 1987, A5(4):1375-84; Hayashi, (1987) Physics Today, December 1987, pp. 44-60; MRS Bulletin, January 1990, pgs. 16-47.

As further described in U.S. Patent Publication No. 20030147966, nanoparticles contemplated are produced using HAuCl₄ and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein. In further embodiments, a plurality of SNAs (e.g., liposomal particles) is produced and the SNAs in the plurality have a mean diameter of less than or equal to about 50 nanometers (e.g., about 5 nanometers to about 50 nanometers, or about 5 nanometers to about 40 nanometers, or about 5 nanometers to about 30 nanometers, or about 5 nanometers to about 20 nanometers, or about 10 nanometers to about 50 nanometers, or about 10 nanometers to about 40 nanometers, or about 10 nanometers to about 30 nanometers, or about 10 nanometers to about 20 nanometers). In further embodiments, the SNAs in the plurality created by a method of the disclosure have a mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers.

Antigen. The present disclosure provides SNAs comprising an antigen. In various embodiments, the antigen is a tumor associated antigen, a tumor specific antigen, or a neo-antigen. n some embodiments, the antigen is OVA1, MSLN, P53, Ras, a melanoma related antigen (e.g., Gp100,MAGE, Tyrosinase), a HPV related antigen (e.g., E6, E7), a prostate cancer related antigen (e.g., PSA, PSMA, PAP, hTARP), an ovarian cancer related antigen (e.g., CA-125), a breast cancer related antigen (e.g., MUC-1, TEA), a hepatocellular carcinoma related antigen (e.g., AFP), a bowel cancer related antigen (e.g., CEA), or human papillomavirus (HPV) E7 nuclear protein. Other antigens are contemplated for use according to the compositions and methods of the disclosure; any antigen for which an immune response is desired is contemplated herein.

It is contemplated herein that an antigen for use in the compositions and methods of the disclosure is attached to a nucleic acid on the surface of a SNA, or attached to the surface of a SNA through a linker as disclosed herein, or both. In some embodiments, an antigen is encapsulated in the SNA in addition to being surface-attached.

Linkers. The disclosure provides compositions and methods in which an antigen is associated with and/or attached to the surface of a SNA via a linker. The linker can be, in various embodiments, a cleavable linker, a non-cleavable linker, a traceless linker, and a combination thereof.

The linker links the antigen to the oligonucleotide in the disclosed SNA (i.e., Antigen-LINKER-Oligonucleotide). The oligonucleotide can be hybridized to another oligonucleotide attached to the SNA or can be directed attached to the SNA (e.g., via attachment to an associative moiety). Some specifically contemplated linkers include carbamate alkylene, carbamate alkylenearyl dithiolate linkers, amide alkylene dithiolate linkers, amide alkylenearyl dithiolate linkers, and amide alkylene succinimidyl linkers. In some cases, the linker comprises —NH—C(O)—O—C₂₋₅alkylene-S—S—C₂₋₇alkylene- or —NH—C(O)—C₂₋₅alkylene-S—S—C₂₋₇alkylene-. The carbon alpha to the —S—S— moiety can be branched, e.g., —CHX—S—S— or —S—S—CHY— or a combination thereof, where X and Y are independently Me, Et, or iPr. The carbon alpha to the antigen can be branched, e.g., —CHX—C₂₋₄alkylene-S—S—, where X is Me, Et, or iPr. In some cases, the linker is —NH—C(O)—O—CH₂—Ar—S—S—C₂₋₇alkylene-, and Ar is a meta- or para-substituted phenyl. In some cases, the linker is —NH—C(O)—C₂₋₄alkylene-N-succinimidyl-S—C₂₋₆alkylene-.

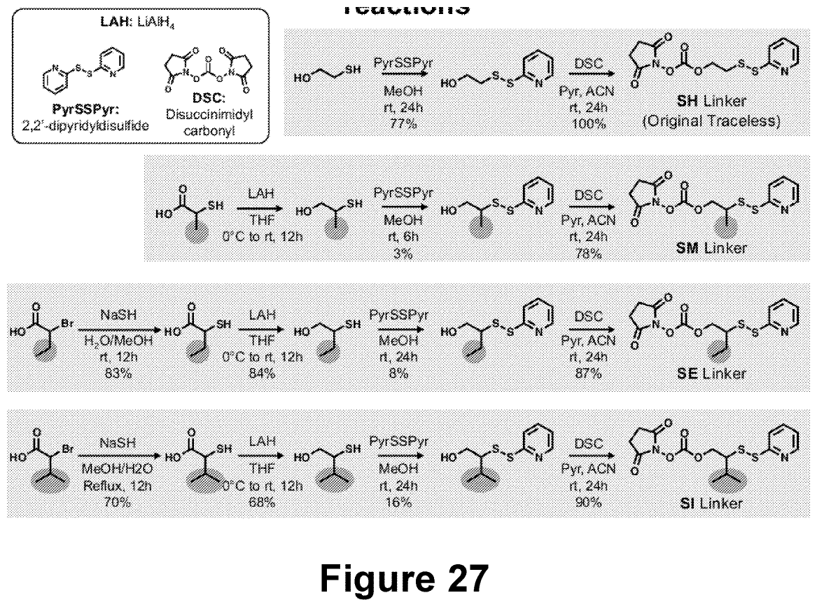

Additional linkers are shown in FIG. 27 (i.e., SH linker, SM linker, SE linker, and SI linker). The disclosure contemplates multiple points of attachment available for modulating antigen release (e.g., disulfide cleavage, linker cyclization, and dehybridization), and the kinetics of antigen release at each attachment point can be controlled. For example, steric bulk about the disulfide can decrease the rate of the S_{N}2 reaction; increased length of an alkyl spacer can affect the rate of ring closure; and mismatched nucleotide sequences lower the melting temperature (Tₘ), while locked nucleic acids increase the Tₘ.

Polynucleotides. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5 ,4-b] [1,4]benzoxazin-2(3H)-one), phenothiazine cytidine (1H-pyrimido[5,4-b] [1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or a modified form thereof generally comprise a polynucleotide from about 5 nucleotides to about 100 nucleotides in length. More specifically, nanoparticles are functionalized with a polynucleotide that is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length are contemplated.

In some embodiments, the polynucleotide attached to a nanoparticle is DNA. When DNA is attached to the nanoparticle, the DNA is in some embodiments comprised of a sequence that is sufficiently complementary to a target region of a polynucleotide such that hybridization of the DNA polynucleotide attached to a nanoparticle and the target polynucleotide takes place, thereby associating the target polynucleotide to the nanoparticle. The DNA in various aspects is single stranded or double-stranded, as long as the double-stranded molecule also includes a single strand region that hybridizes to a single strand region of the target polynucleotide. In some aspects, hybridization of the polynucleotide functionalized on the nanoparticle can form a triplex structure with a double-stranded target polynucleotide. In another aspect, a triplex structure can be formed by hybridization of a double-stranded oligonucleotide functionalized on a nanoparticle to a single-stranded target polynucleotide.

In some embodiments, the disclosure contemplates that a polynucleotide attached to a nanoparticle is RNA. The RNA can be either single-stranded or double-stranded, so long as it is able to hybridize to a target polynucleotide.

In some aspects, multiple polynucleotides are functionalized to a nanoparticle. In various aspects, the multiple polynucleotides each have the same sequence, while in other aspects one or more polynucleotides have a different sequence. In further aspects, multiple polynucleotides are arranged in tandem and are separated by a spacer. Spacers are described in more detail herein below.

Polynucleotide attachment to a nanoparticle. Polynucleotides contemplated for use in the methods include those bound to the nanoparticle through any means (e.g., covalent or non-covalent attachment). Regardless of the means by which the polynucleotide is attached to the nanoparticle, attachment in various aspects is effected through a 5′ linkage, a 3′ linkage, some type of internal linkage, or any combination of these attachments. In some embodiments, the polynucleotide is covalently attached to a nanoparticle. In further embodiments, the polynucleotide is non-covalently attached to a nanoparticle. An oligonucleotide of the disclosure comprises, in various embodiments, an associative moiety selected from the group consisting of a tocopherol, a cholesterol moiety, DOPE-butamide-phenylmaleimido, and lyso-phosphoethanolamine-butamide-pneylmaleimido. See also U.S. Patent Application Publication No. 2016/0310425, incorporated by reference herein in its entirety.

Methods of attachment are known to those of ordinary skill in the art and are described in US Publication No. 2009/0209629, which is incorporated by reference herein in its entirety. Methods of attaching RNA to a nanoparticle are generally described in PCT/US2009/65822, which is incorporated by reference herein in its entirety. Methods of associating polynucleotides with a liposomal particle are described in PCT/US2014/068429, which is incorporated by reference herein in its entirety.

Spacers. In certain aspects, functionalized nanoparticles are contemplated which include those wherein an oligonucleotide is attached to the nanoparticle through a spacer. “Spacer” as used herein means a moiety that does not participate in modulating gene expression per se but which serves to increase distance between the nanoparticle and the functional oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, an ethylglycol, or combinations thereof.

In certain aspects, the polynucleotide has a spacer through which it is covalently bound to the nanoparticles. These polynucleotides are the same polynucleotides as described above. As a result of the binding of the spacer to the nanoparticles, the polynucleotide is spaced away from the surface of the nanoparticles and is more accessible for hybridization with its target. In various embodiments, the length of the spacer is or is equivalent to at least about 5 nucleotides, 5-10 nucleotides, 10 nucleotides, 10-30 nucleotides, or even greater than 30 nucleotides. The spacer may have any sequence which does not interfere with the ability of the polynucleotides to become bound to the nanoparticles or to the target polynucleotide. In certain aspects, the bases of the polynucleotide spacer are all adenylic acids, all thymidylic acids, all cytidylic acids, all guanylic acids, all uridylic acids, or all some other modified base.

Nanoparticle surface density. A surface density adequate to make the nanoparticles stable and the conditions necessary to obtain it for a desired combination of nanoparticles and polynucleotides can be determined empirically. Generally, a surface density of at least about 2 pmoles/cm² will be adequate to provide stable nanoparticle-oligonucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm2, at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Alternatively, the density of polynucleotide on the surface of the SNA is measured by the number of polynucleotides on the surface of a SNA. With respect to the surface density of polynucleotides on the surface of a SNA of the disclosure, it is contemplated that a SNA as described herein comprises from about 1 to about 100 oligonucleotides on its surface. In various embodiments, a SNA comprises from about 10 to about 100, or from 10 to about 90, or from about 10 to about 80, or from about 10 to about 70, or from about 10 to about 60, or from about 10 to about 50, or from about 10 to about 40, or from about 10 to about 30, or from about 10 to about 20 oligonucleotides on its surface. In further embodiments, a SNA comprises at least about 5, 10, 20, 30, 40, 45, 50, 55, 60, 65, 70, 75, 80, 85, 90, 95, or 100 polynucleotides on its surface.

### Uses of SNAs in Gene Regulation/Therapy

In addition to serving a role in providing an oligonucleotide (e.g., an immunostimulatory oligonucleotide) and an antigen to a cell, it is also contemplated that in some embodiments, a SNA of the disclosure possesses the ability to regulate gene expression. Thus, in some embodiments, a SNA of the disclosure comprises an antigen that is associated with a SNA through a linker, an oligonucleotide (e.g., an immunostimulatory oligonucleotide), and an additional oligonucleotide having gene regulatory activity (e.g., inhibition of target gene expression or target cell recognition). Accordingly, in some embodiments the disclosure provides methods for inhibiting gene product expression, and such methods include those wherein expression of a target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide.

In various aspects, the methods include use of an oligonucleotide which is 100% complementary to the target polynucleotide, i.e., a perfect match, while in other aspects, the oligonucleotide is about or at least (meaning greater than or equal to) about 95% complementary to the polynucleotide over the length of the oligonucleotide, about or at least about 90%, about or at least about 85%, about or at least about 80%, about or at least about 75%, about or at least about 70%, about or at least about 65%, about or at least about 60%, about or at least about 55%, about or at least about 50%, about or at least about 45%, about or at least about 40%, about or at least about 35%, about or at least about 30%, about or at least about 25%, about or at least about 20% complementary to the polynucleotide over the length of the oligonucleotide to the extent that the oligonucleotide is able to achieve the desired degree of inhibition of a target gene product. Moreover, an oligonucleotide may hybridize over one or more segments such that intervening or adjacent segments are not involved in the hybridization event (e.g., a loop structure or hairpin structure). The percent complementarity is determined over the length of the oligonucleotide. For example, given an inhibitory oligonucleotide in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Accordingly, methods of utilizing a SNA of the disclosure in gene regulation therapy are provided. This method comprises the step of hybridizing a polynucleotide encoding the gene with one or more oligonucleotides complementary to all or a portion of the polynucleotide, the oligonucleotide being the additional oligonucleotide of a composition as described herein, wherein hybridizing between the polynucleotide and the additional oligonucleotide occurs over a length of the polynucleotide with a degree of complementarity sufficient to inhibit expression of the gene product. The inhibition of gene expression may occur in vivo or in vitro.

The oligonucleotide utilized in the methods of the disclosure is either RNA or DNA. The RNA can be an inhibitory RNA (RNAi) that performs a regulatory function, and in various embodiments is selected from the group consisting of a small inhibitory RNA (siRNA), an RNA that forms a triplex with double stranded DNA, and a ribozyme. Alternatively, the RNA is microRNA that performs a regulatory function. The DNA is, in some embodiments, an antisense-DNA.

### Use of SNAs in Immune Regulation

Toll-like receptors (TLRs) are a class of proteins, expressed in sentinel cells, that plays a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 4, TLR 8 and TLR 9 that respond to specific oligonucleotide are located inside special intracellular compartments, called endosomes. The mechanism of modulation of TLR 4, TLR 8 and TLR9 receptors is based on DNA-protein interactions.

Synthetic immunostimulatory oligonucleotides that contain CpG motifs that are similar to those found in bacterial DNA stimulate a similar response of the TLR receptors. Therefore immunomodulatory oligonucleotides have various potential therapeutic uses, including treatment of immune deficiency and cancer.

In some embodiments, the disclosure provides a method of up-regulating activity of a TLR comprising contacting a cell having the TLR with a SNA of the disclosure. In further embodiments, the cell is an antigen presenting cell (APC). In some embodiments, the APC is a dendritic cell, while in further embodiments the cell is a leukocyte. The leukocyte, in still further embodiments, is a phagocyte, an innate lymphoid cell, a mast cell, an eosinophil, a basophil, a natural killer (NK) cell, a T cell, or a B cell. The phagocyte, in some embodiments, is a macrophage, a neutrophil, or a dendritic cell.

Down regulation of the immune system would involve knocking down the gene responsible for the expression of the Toll-like receptor. This antisense approach involves use of SNAs conjugated to specific antisense oligonucleotide sequences to knock down the expression of any toll-like protein.

Accordingly, methods of utilizing SNAs for modulating toll-like receptors are disclosed. The method either up-regulates or down-regulates the Toll-like-receptor through the use of a TLR agonist or a TLR antagonist, respectively. The method comprises contacting a cell having a toll-like receptor with a SNA of the disclosure. The toll-like receptors modulated include toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13.

Compositions. The disclosure includes compositions that comprise a pharmaceutically acceptable carrier and a spherical nucleic acid (SNA) of the disclosure, wherein the SNA comprises a nanoparticle, an oligonucleotide on the surface of the nanoparticle, and an antigen that is associated with the surface of the SNA via a linker. In some embodiments, the composition is an antigenic composition. The term “carrier” refers to a vehicle within which the SNA is administered to a mammalian subject. The term carrier encompasses diluents, excipients, adjuvants and combinations thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers (e.g., phosphate, tris, borate, succinate, or histidine). Exemplary “excipients” are inert substances include but are not limited to polymers (e.g., polyethylene glycol), carbohydrates (e.g., starch, glucose, lactose, sucrose, or cellulose), and alcohols (e.g., glycerol, sorbitol, or xylitol).

Adjuvants are include but are not limited to emulsions, microparticles, immune stimulating complexes (iscoms), LPS, CpG, or MPL.

Methods of inducing an immune response. The disclosure includes methods for eliciting an immune response in a subject in need thereof, comprising administering to the subject an effective amount of a composition or vaccine of the disclosure. In some embodiments, the vaccine is a cancer vaccine. In further embodiments, the cancer is selected from the group consisting of bladder cancer, breast cancer, colon and rectal cancer, endometrial cancer, glioblastoma, kidney cancer, leukemia, liver cancer, lung cancer, melanoma, non-hodgkin lymphoma, osteocarcinoma, ovarian cancer, pancreatic cancer, prostate cancer, thyroid cancer, and human papilloma virus-induced cancer.

The immune response raised by the methods of the present disclosure generally includes an innate and adaptive immune response, preferably an antigen presenting cell response and/or CD8⁺ and/or CD4⁺ T-cell response and/or antibody secretion (e.g., a B-cell response). The immune response generated by a composition as disclosed herein is directed against, and preferably ameliorates and/or neutralizes and/or reduces the tumor burden of cancer. Methods for assessing immune responses after administration of a composition of the disclosure (immunization or vaccination) are known in the art and/or described herein. Antigenic compositions can be administered in a number of suitable ways, such as intramuscular injection, subcutaneous injection, intradermal administration and mucosal administration such as oral or intranasal. Additional modes of administration include but are not limited to intranasal administration, and oral administration.

Antigenic compositions may be used to treat both children and adults. Thus a subject may be less than 1 year old, 1-5 years old, 5-15 years old, 15-55 years old, or at least 55 years old.

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, or a mucosal prime and parenteral boost. Administration of more than one dose (typically two doses) is particularly useful in immunologically naive subjects or subjects of a hyporesponsive population (e.g., diabetics, or subjects with chronic kidney disease). Multiple doses will typically be administered at least 1 week apart (e.g., about 2 weeks, about 3 weeks, about 4 weeks, about 6 weeks, about 8 weeks, about 10 weeks, about 12 weeks, or about 16 weeks). Preferably multiple doses are administered from one, two, three, four or five months apart. Antigenic compositions of the present disclosure may be administered to patients at substantially the same time as (e.g., during the same medical consultation or visit to a healthcare professional) other vaccines.

Articles of Manufacture and Kits. The disclosure additionally includes articles of manufacture and kits comprising a composition described herein. In some embodiments, the kits further comprise instructions for measuring antigen-specific antibodies. In some embodiments, the antibodies are present in serum from a blood sample of a subject immunized with a composition comprising a SNA of the disclosure.

As used herein, the term “instructions” refers to directions for using reagents contained in the kit for measuring antibody titer. In some embodiments, the instructions further comprise the statement of intended use required by the U.S. Food and Drug Administration (FDA) in labeling in vitro diagnostic products.

The following examples illustrate various embodiments contemplated by the present disclosure. The examples are exemplary in nature and are in no way intended to be limiting.

### EXAMPLES

### Example 1

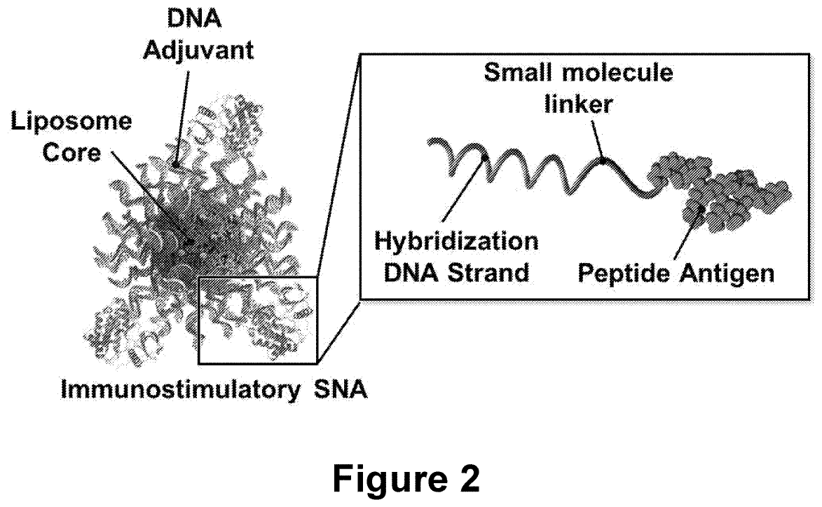

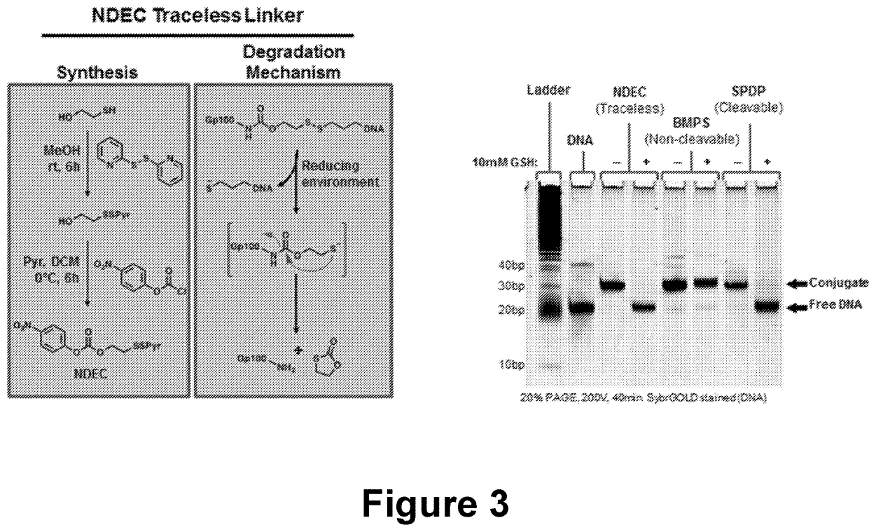

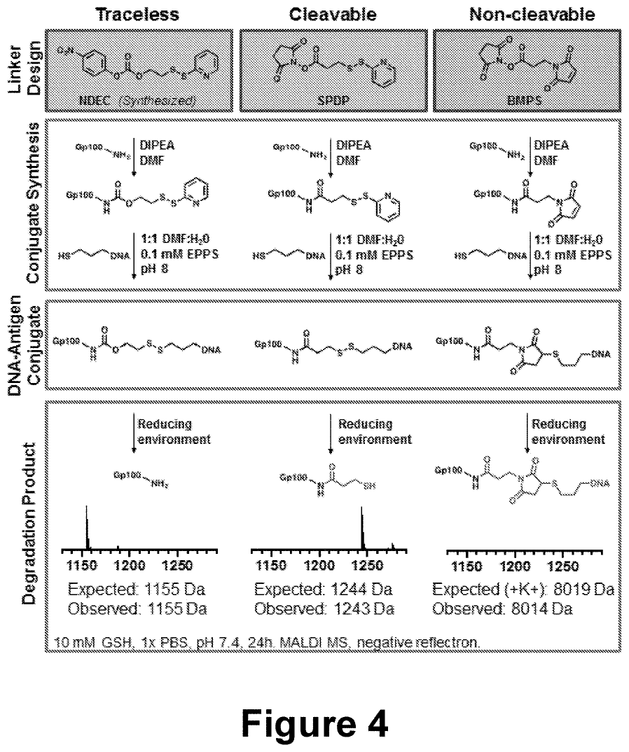

In some embodiments of the disclosure, an antigen is attached to a DNA strand that is hybridized to the surface of an SNA (FIG. 2). As disclosed herein, the linker chemistry chosen for the conjugation to a DNA strand affects the chemical structure of the antigen delivered to an APC cell. Since T-cell response is dependent on the structure of the antigen, which must bind to MHC and TCR, conjugation chemistry is an important design consideration for immunostimulatory SNAs. In various embodiments, the linker chemistry utilized on a given SNA is non-cleavable, cleavable, and/or traceless (FIG. 3). FIG. 4 depicts shows the linker design, conjugate synthesis, DNA-Antigen conjugate structure, and degradation product for three linker designs.

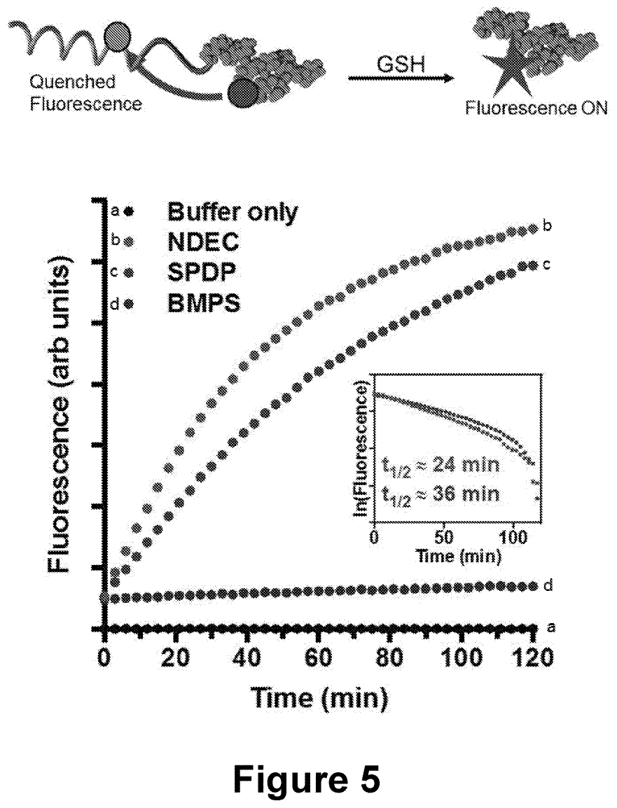

FIG. 5 shows the kinetics of linker cleavage in the presence of 10 mM GSH. Both cleavable linker conjugates, NDEC and SPDP, showed an increase in fluorescence corresponding to a half-life of approximately 24 and 36 minutes, respectively. Thus, linker half-life is less than one hour at cytosolic conditions.

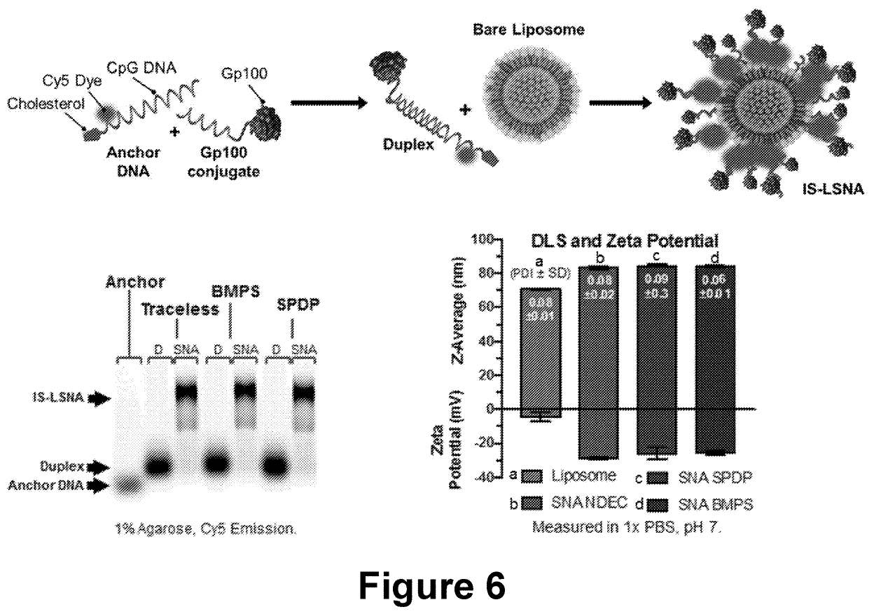

FIG. 6 depicts examples of spherical nucleic acid synthesis and characterization, including changes in electrophoretic mobility, hydrodynamic radius, and zeta potential indicate formation of monodisperse SNAs. Compared to bare liposomes, the Z-average hydrodynamic diameter of particles increased by approximately 13 nm and the Zeta potential decreased by approximately 22 mV. All the anchor strands are associated with the liposomal core, indicated by a lack of band corresponding to free anchor in the agarose gel.

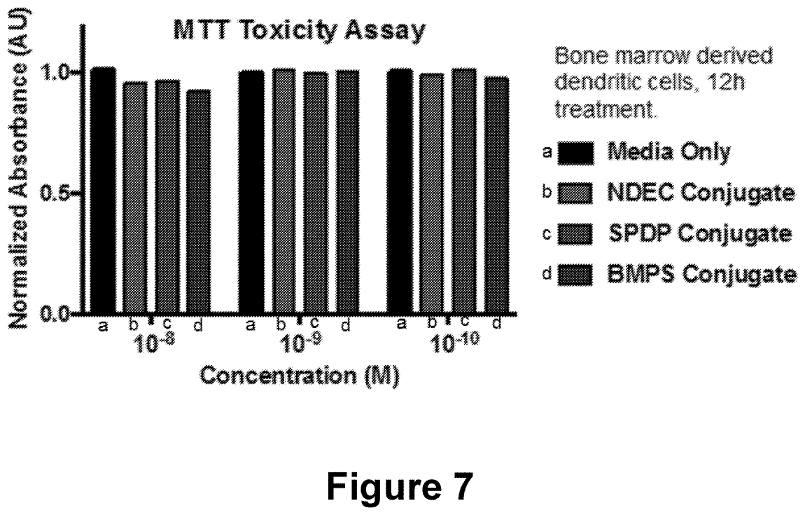

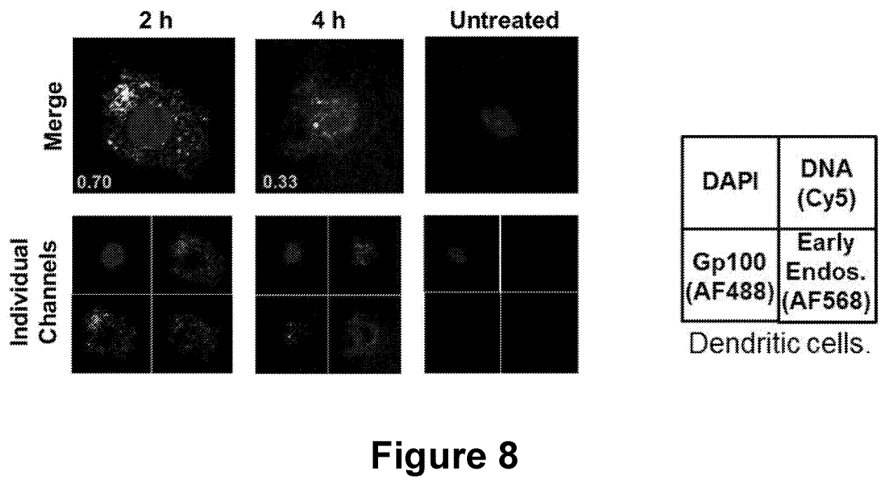

FIG. 7 shows that no significant toxicity was observed by MTT assay using Dendritic cells with any of the three SNAs made with different linker conjugates. FIG. 8 shows that SNAs deliver both adjuvant and antigen to dendritic cells. SNAs deliver both adjuvant CpG motif DNA (tagged with Cy5) and antigen gp100 peptide (tagged with AlexaFluor 488). The co-delivery efficiency is higher for adjuvant, antigen pairs formulated as an SNA compared to free in solution mixture.

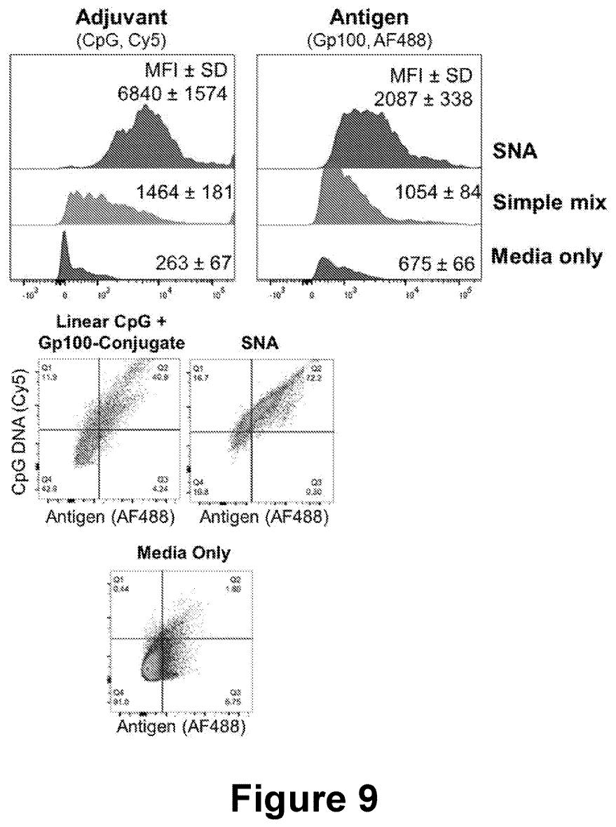

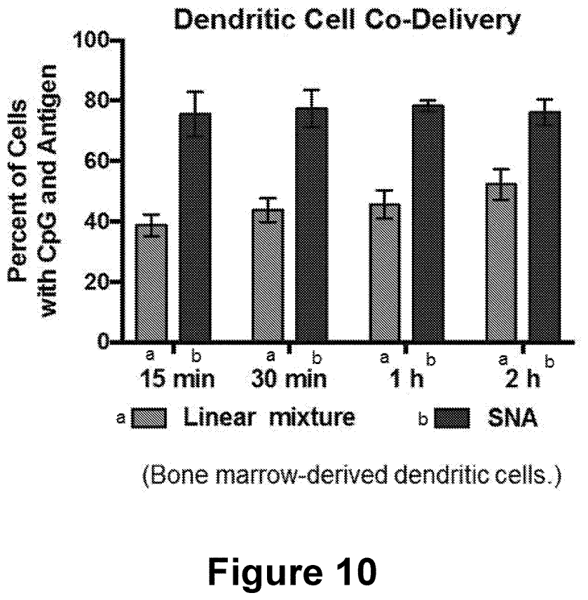

FIG. 9 shows: Top two panels show the delivery of Cy5-labled adjuvant (CpG) and AF488-labled antigen (gp100) is more efficient in an SNA form compared to a simple mixture of the two components. The bottom panel shows that the co-delivery efficiency of adjuvant and antigen are more efficient for SNAs compared to a simple mixture of the two components. This is representative data of FIG. 10. FIG. 10 shows higher co-delivery of antigen and adjuvant in dendritic cells when they are structured in an SNA architecture compared to a simple mixture of the two components.

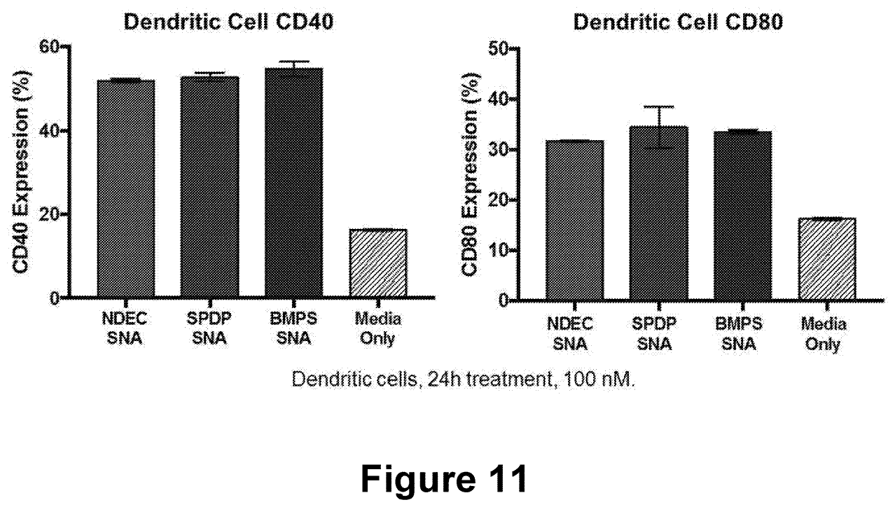

FIG. 11 shows that dendritic cell activation markers, CD40 and CD80, were upregulated compared to a media-only control. The upregulation was indistinguishable between all linker types. This result indicated that the differences in linker chemistry do not significantly impact DC activation.

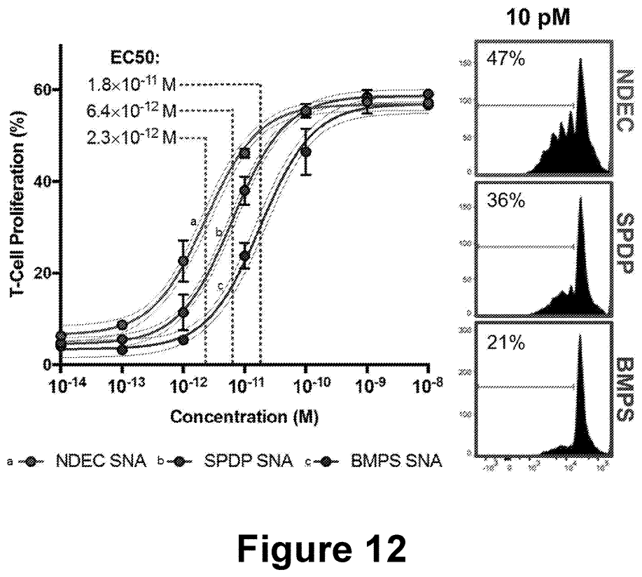

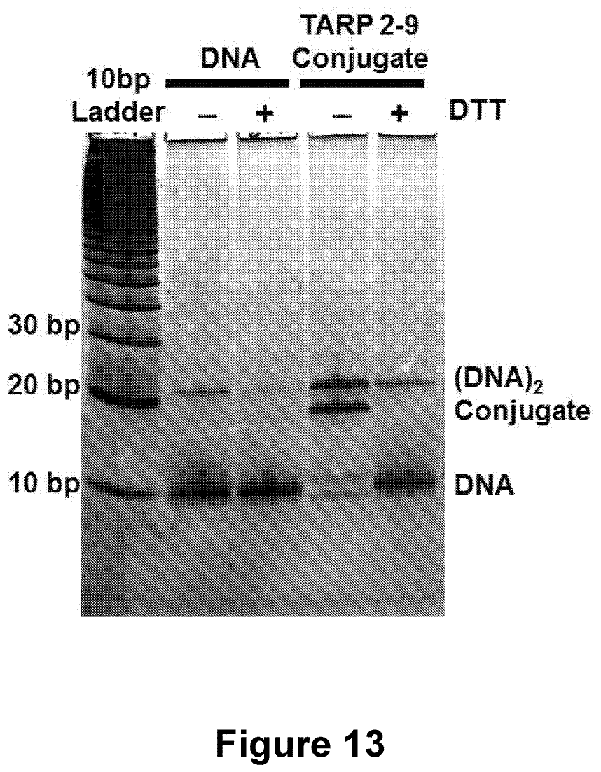

FIG. 12 shows that the potency of immunostimulatory SNAs, as measured by T-Cell proliferation, is affected by linker chemistry. Traceless linker (NDEC) provided a nearly eight-fold increase in potency as measured by EC₅₀ over the non-cleavable linker chemistry (BMPS), and a nearly three-fold increase over the cleavable but non-traceless counterpart (SPDP). Each measurement is an average of three technical replicates, standard deviations shown (left panel). A three parameter logistic dose-response curve was used to fit the data, 95% confidence bands of the fit are shaded. Set of chosen replicates of flow cytometry data at the 10 pM concentration is shown for the three linker types (right panel).

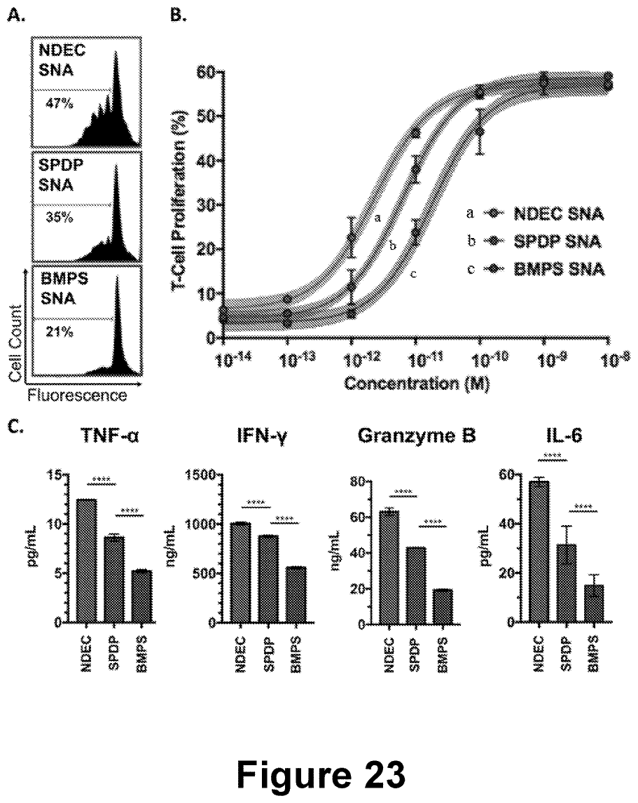

T-cell activation is measured by quantifying amount of cytokines (IL-2, IFN-γ) released into the media. See FIG. 23. In addition, TARP peptides are used to study prostate cancer in a humanized-mouse model. Conjugates are synthesized with multiple TARP peptides as well as E7.

### Example 2

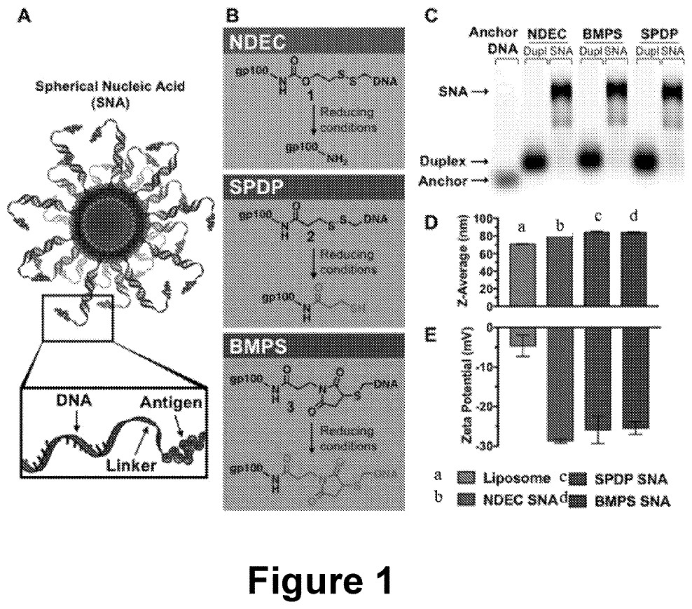

The use of three linkage types was demonstrated—a disulfide reduction-activated traceless linker, a disulfide reduction-activated cleavable linker, and a non-cleavable linker (FIG. 1A,B)—for attaching a human melanoma-specific antigenic peptide, gp100, to SNAs. The study was designed to probe the importance, or lack thereof, of generating pristine antigens for immune activation. The gp100 melanoma antigen (KVPRNQDWL) (SEQ ID NO: 1) was chosen as a model system because of its clinical relevance to human diseases and high potential for translation.¹⁷

The data show that while the antigen chemistry did not impede TLR-9 regulated APC activation, it significantly augmented the downstream T-cell response in terms of both activation and proliferation. A comparison of three linker types, 1) non-cleavable, 2) cleavable but non-traceless, and 3) traceless, revealed up to an eight-fold improvement in T-cell proliferation, when the traceless linker is used. This work underscored the critical importance of the choice of conjugation chemistry in vaccine development.

Immunostimulatory SNAs were synthesized using a liposomal core with TLR9-stimulatory CpG B oligonucleotides (see Table 1 for sequences), tagged with a Cy5 dye, and immobilized on the core surface through intercalation by using a cholesterol anchor on the 3′ end.¹⁸⁻¹⁹ Antigens were attached to the SNA as one of three gp100-DNA conjugate types, 1-3, made with DNA complementary to the CpG adjuvant. CpG anchor stands were all hybridized to the conjugates prior to their addition to liposomes, these duplexes were added at a 75:1 ratio to liposomes. All design parameters, such as the 1:1 ratio of antigen to adjuvant, DNA and gp100 concentrations were kept constant across the SNA structures investigated—only the identity of the linker differed.

TABLE 1
Oligonucleotide sequences used in the studies.

| | |
|---|---|
| CpG Anchor (PS) | 5′-TCC ATG ACG TTC CTG ACG TT |
| | (Cy5) (Sp18)₂ Cholesterol-3′ |
| | (SEQ ID NO: 2) |
| Conjugate | 5′-AAC GTC AGG AAC GTC ATG GA |
| (CpG | Sp18 C3Thiol-3′ |
| complement, PO) | (SEQ ID NO: 3) |
| FRET | 5′-AAC GTC AGG AAC GTC ATG GA |
| conjugate (PO) | (Sp18) (Eclipse Quencher) |
| | C3Thiol-3′ |
| | (SEQ ID NO: 4) |

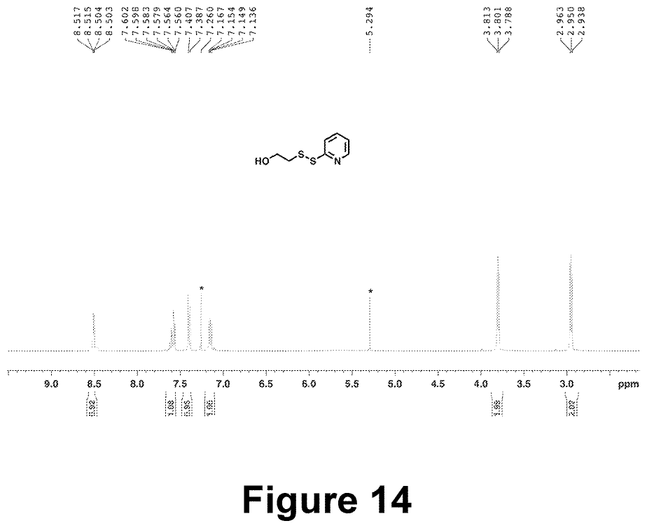

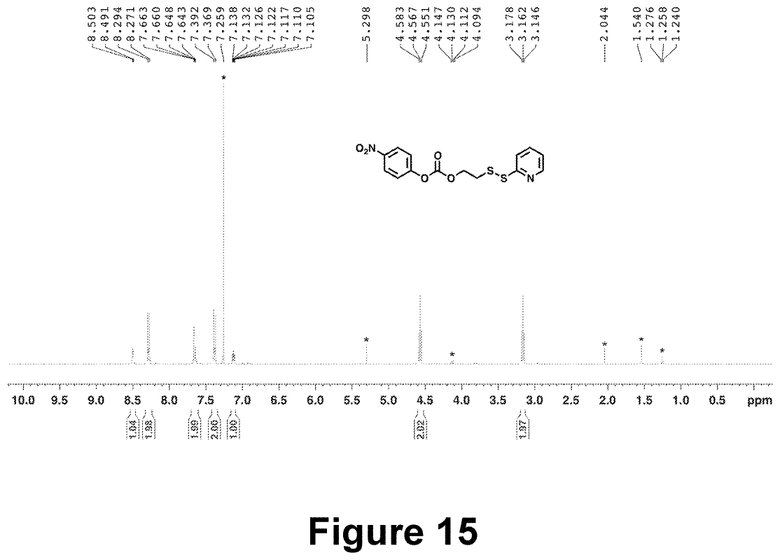

Conjugates 1-3 were synthesized by first attaching one end of the linker to a peptide amine, followed by attachment of thiolated DNA to the other. The amine residue of the antigen was used as a chemical point for conjugation since this strategy can be adapted to other antigens, all of which have at least one primary amine at their N-terminus. The three distinct linker chemistries were chosen for antigen attachment (FIG. 1B). A commercially available non-cleavable linker (N—(β-maleimidopropyloxy) succinimide ester, BMPS) was used to create conjugate 3, which has no readily-cleavable bonds. A commercially available cleavable linker (succinimidyl 3-(2-pyridyldithio)propionate, SPDP) was used to prepare conjugate 2, which cleaves in the reducing environment of the cell but leaves a molecular pendant group (3-mercaptopropionamide) attached to the antigen. Finally, a traceless linker (4-nitrophenyl 2-(2-pyridyldithio)ethyl carbonate, NDEC),^{15-16, 20-22} was incorporated to create conjugate 1 (See FIGS. 14-15 for NMR spectra). The traceless linker incorporates a disulfide, which upon reduction, results in an intramolecular cyclization that releases the antigen in an unmodified form.

2-(2-Pyridinyldisulfanyl)ethanol. 2-mercaptoethanol (2.24 g, 28.7 mmol) was added to a solution of 2,2′-dipyridyldisulfide (9.49 g, 43.1 mmol) in methanol (30 mL). The mixture was stirred at room temperature for 12 hours, then the reaction solvent was evaporated under reduced pressure and reconstituted in dichloromethane. The solution was washed with 10% sodium hydroxide in water and saturated sodium chloride solution. The product was purified on a silica gel column with diethyl ether/hexanes solvent system and isolated as a yellow oil (4.13 g, 77%). ¹H NMR (400 MHz, CDCl₃, δ): 8.51 (ddd, J=5.0, 1.9, 1.0 Hz, 1H), 7.58 (ddd, J=8.1, 7.4, 1.8 Hz, 1H), 7.40 (m, 1H), 7.15 (ddd, J=7.4, 4.9, 1.0 Hz, 1H), 3.80 (t, 2H, J=5.1 Hz), 2.95 (t, 2H, J=5.1). See FIG. 14.

4-Nitrophenyl 2-(2-pyridyldithio)ethyl carbonate (NDEC). 2-(2-Pyridinyldisulfanyl)ethanol (2.72 g, 14.5 mmol) was combined with triethylamine (2.23 mL, 16.0 mmol) in anhydrous dichloromethane under a nitrogen atmosphere. 4-Nitrophenyl chloroformate (3.5 g, 17.4 mmol) was added to the solution and left to stir at room temperature overnight. Solvent was removed under reduced pressure and the crude mixture was purified using silica chromatography with ethyl acetate/dichloromethane solvent system. The product was isolated as a yellow oil (1.75 g, 25%). ¹H NMR (400 MHz, CDCl₃, δ): 8.50 (m, 1H), 8.28 (m, 2H), 7.65 (m, 2H), 7.38 (m, 2H), 7.12 (ddd, J=6.4, 4.8, 2.1 Hz, 1H), 4.57 (t, 2H, J=6.4 Hz), 3.16 (t, 2H, J=6.4 Hz). See FIG. 15.

General procedure for synthesis of gp100-DNA conjugates. To prepare gp100-DNA conjugates, linkers were attached to the gp100 peptide first, followed by attachment of thiol-modified DNA. The gp100 peptide (5.8 mg, 5 μmol) was dissolved in anhydrous dimethyl formamide (200 μL) to which was added the linker (1 μmol) and diisopropylethylamine (5 μmol). The reaction was shaken at room temperature overnight. Afterwards, the peptide product was precipitated and washed thrice with 2 mL of 5% acetic acid in diethyl ether. The remaining acetic acid and diethyl ether were evaporated under reduced pressure.

In order to add the DNA, the crude gp100-linker conjugate was combined with thiol modified DNA (1 equivalent) in 400 μL of 1:1 solution of water:dimethylformamide and 0.1 M EPPS buffer at pH 8.0. The reaction was shaken overnight at room temperature. Following this, the reaction was diluted with water and washed five times with water using an Amicon 3 kDa-0.5 mL molecular weight cut-off filter. This product was purified using denaturing polyacrylamide gel electrophoresis, and further washed eight times with water using an Amicon 3 kDa-15mL filter. (32% yield over two steps with respect to limiting reagent)

AlexaFluor 488-modified conjugates were synthesized as described above, then incubated with NHS-ester activated AlexaFluor dye (10 equiv.) for 12 hours and purified by washing ten times with water in a molecular weight cut-off filter (3 kDa-0.5mL, Amicon).

General procedure for SNA synthesis. SNA synthesis was carried out in three independent steps: duplex formation, liposome synthesis, and SNA assembly. To form duplex strands, the gp100-DNA conjugate was mixed with an equimolar amount of complementary strand labeled with Cy5 and bearing a 3″-cholesterol group. The solution was lyophilized and reconstituted in buffer (1× duplex buffer, IDT) to a concentration of 200 μM by duplex. This solution was heated to 70° C., allowed to cool to room temperature and incubated at 4° C. overnight.

Liposomes were synthesized by drying a film of 50 mg of DOPC in chloroform (Avanti Polar Lipid 850375C) in a glass vial using dry nitrogen gas followed by overnight lyophilization. The phospholipids were hydrated with 5 mL of PBS followed by vortexing and five freeze/thaw cycles, followed by extrusion through 200 nm, 100 nm, 80 nm and 50 nm polycarbonate filters, consecutively (Sterlitech). After concentration, diameters of liposomes were measured by DLS using a Zetasizer Nano ZS.

SNAs were assembled by mixing the duplex with liposomes in a 75:1 ratio and diluting with 1×PBS to a concentration of 100 μM by duplex (or 0.133 μM by SNA). The solution was shaken at 33° C. overnight and then used without further purification. It was assumed that linker identity did not impact cholesterol anchor intercalation into the liposome and thus the SNA loading.

Quantification of cleavage kinetics. Cleavage kinetics of the conjugate were quantified by incubating conjugates 1-3 at 200 nM concentration in 1×PBS with 20 mM GSH at room temperature, and monitoring the emission at 520 nm while exciting at 485 nm. No increase in fluorescence was observed for the samples incubated in PBS only, while samples 1 and 2 showed increase in fluorescence in the presence of GSH. Following 90 minutes of incubation, TCEP was added to the reactions, to a 9 mM concentration, to reduce any remaining disulfides and establish a fully-cleaved maximum fluorescence. The PBS only samples served to correct for background fluorescence. A non-linear exponential decay, with the plateau constrained to zero, was used to fit the data and extract the half-lives of 31 (30-32) and 54 (53-56) minutes (95% Cl) for 1 and 2, respectively. The small difference in rates of cleavage is expected to be biologically insignificant and not account for the observed differences in T-cell proliferation.

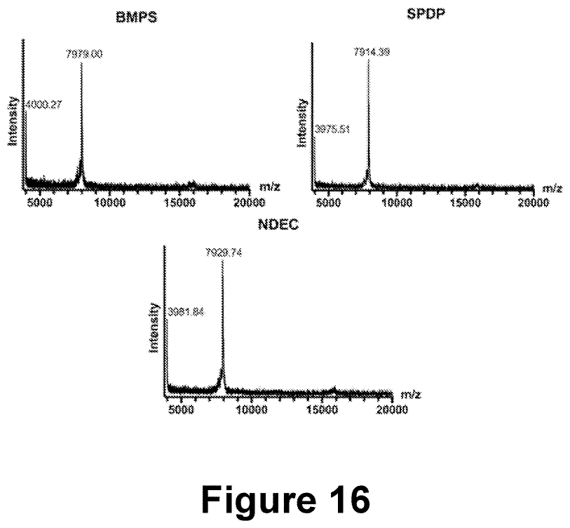

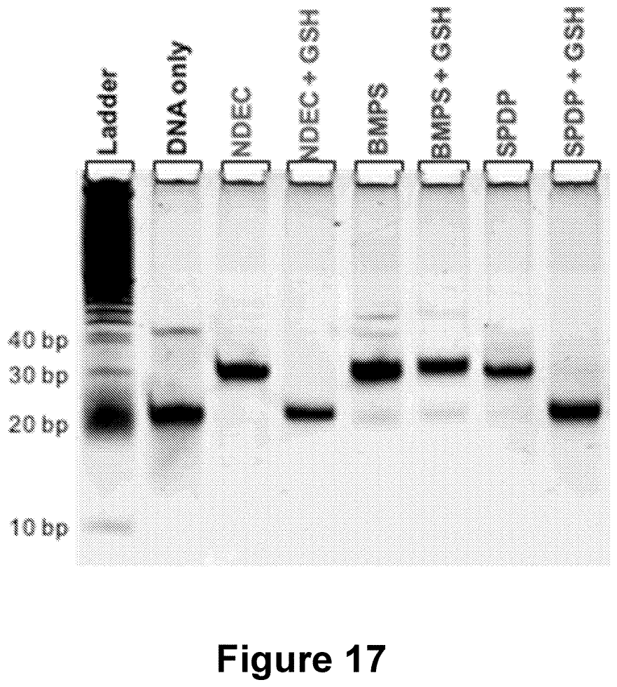

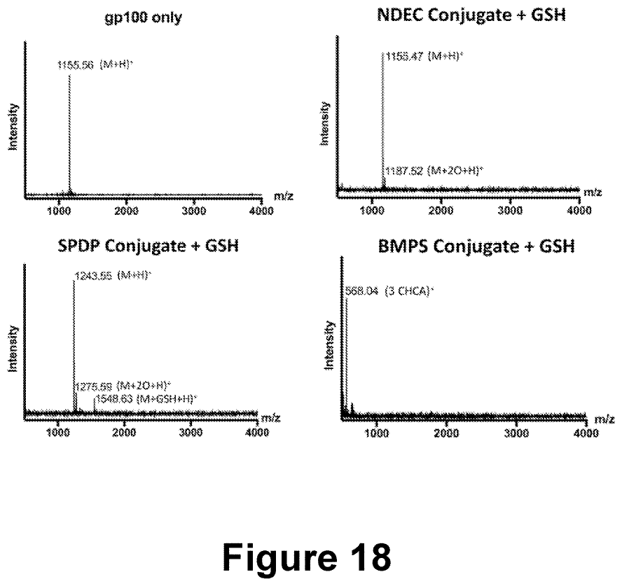

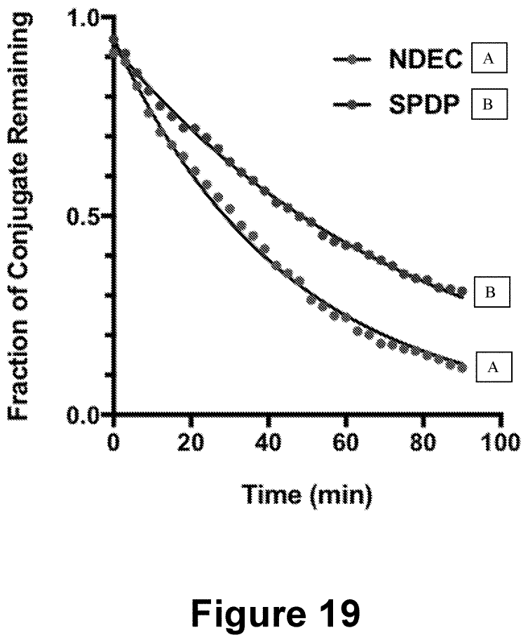

Conjugates 1-3 at pH 7.4 were incubated with 10 mM glutathione and the decomposition products were characterized using PAGE and MALDI-MS. These experiments confirmed that, under cell-mimicking reduction conditions,²³ the BMPS conjugate 3 does not release an antigen, the SPDP conjugate 2 releases an antigen that is modified with a chemical pendant, and the NDEC conjugate 1 regenerates an unmodified gp100 peptide (see FIGS. 16-18). The rate of conjugate cleavage was also characterized by synthesizing 1-3 using a fluorescein labeled gp100 peptide and a quencher-containing oligonucleotide to form a FRET reporter. The fluorescence of this reporter increased upon cleavage of the linkage between the peptide and DNA. Conjugates 1 and 2 were found to have similar cleavage half-lives of approximately 31 and 54 minutes in 20 mM GSH. Conjugate 3 did not show an increase in fluorescence (FIG. 19).

SNAs synthesized with the three conjugates were characterized by agarose gel electrophoresis. A shift in electrophoretic mobility was observed between the single stranded CpG DNA, the duplex with gp100-DNA conjugate, and the SNA (FIG. 1C). Additionally, the SNAs all have indistinguishable z-average hydrodynamic diameter, of 83.7±0.4 nm (PDI 0.075±0.012). An increase of approximately 13 nm over the bare liposomes (FIG. 1D). The zeta potentials of the SNAs were on average −26.7±1.7 mV, a decrease of approximately 20 mV compared to the bare liposomes, which was attributed to the added negative charge carried by the DNA backbone (FIG. 1E).

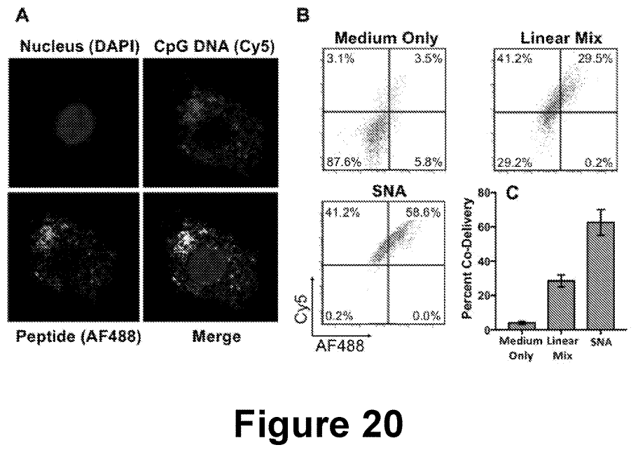

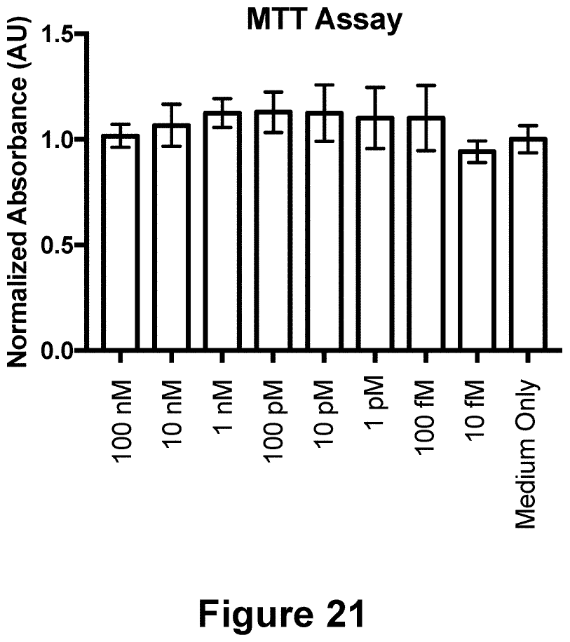

Co-delivery of both adjuvant and antigen is crucial for efficient T-cell activation.²⁴ In order to characterize the co-delivery of these components, bone marrow-derived dendritic cells (DCs) were used as a model system, since they are the most effective professional APCs of the immune system.²⁵ Confocal microscopy images showed that both the AlexaFluor488 (AF488)-labeled gp100 antigen (green) and Cy5-labeled CpG adjuvant (red) were internalized by DCs after incubation with 1-SNAs for fifteen minutes (FIG. 20A). The co-delivery of these components was further quantified using flow cytometry (FIG. 20B). The SNA architecture formulation resulted in a doubling of co-delivery efficiency (double positive of AF488 and Cy5) compared to the linear mixture, as measured over background fluorescence control (medium only) (FIG. 20C). In addition, no significant effect of 1-SNA on cell viability was observed at concentrations below 1 μM using an MTT assay (FIG. 21).

Toxicity Assay. Cytotoxicity of the NDEC-conjugate was assayed using an MTT cell proliferation kit (Roche, Cat. No 11465007001) to ensure that the released linker degradation products were not cytotoxic. Dendritic cells isolated from mice bone marrow were selected by Biotin positive selection kit (Stem Cell Catalog # 18556) and plated in a 96 well-plate with 1×10⁴ cell fluency. Then cells were incubated with SNAs at different concentration for 24 hours at 37° C. and 5% CO₂. Measurements were carried out according to the manufacturer's instructions. No significant toxicity was observed at the tested conditions. Results are shown in FIG. 21, error bars show standard deviations of three replicates.

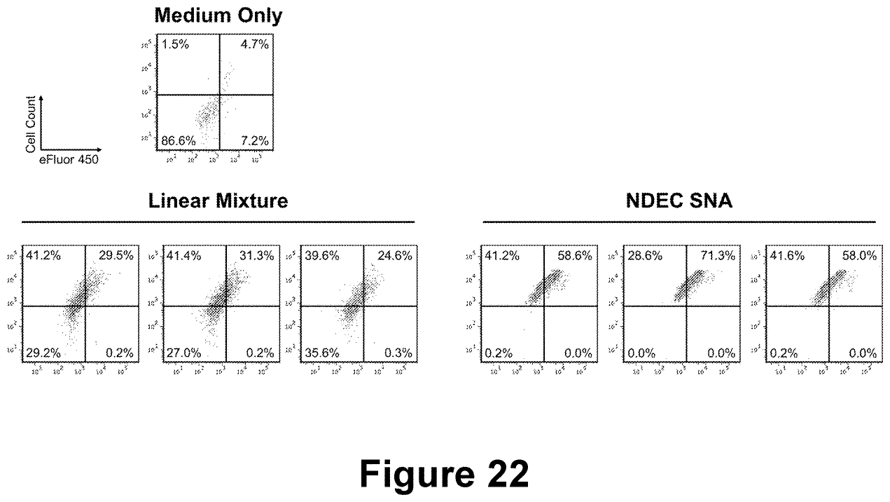

Co-delivery. To measure the uptake of SNAs compared with linear counterpart, bone marrow-derived dendritic cells (DCs) were used that were cultured and stimulated by GM-CSF for 6 days. After that, biotin-positive selection kit (Stem Cell Catalog # 18556) were used to select DCs with the CD11c marker. Then 5E5 cells were treated NDEC SNAs or a linear mixture in an incubator (37° C., 5% CO₂) for 15 minutes before measuring gp100 and CpG uptake. Flow cytometry data shows that SNAs achieve higher uptake measurements compared with linear mixtures. All experiments were performed in triplicate. See FIG. 22.

T-cell receptor transgenic CD8⁺ T-cells (from pmel-1 mice) specifically recognizing gp100 were used to study the efficacy of the immunostimulatory SNAs at eliciting gp100-specific CD8⁺ T-cell responses.²⁶ The splenocytes from pmel-1 mice were treated with each SNA individually at different concentrations for 72 hours to determine a dose-response curve (FIG. 23A, B).²⁷⁻²⁸ It was observed that CD⁸⁺ T-cell proliferation (eFluor 450 dilution) was dependent upon linkage type, the only parameter that differs across the three SNAs. The extent of proliferation was similar across the three structures when splenocytes were treated at the highest concentration range (1-10 nM in gp100), however, at lower concentrations, the T-cell proliferation differed significantly among the three treatment groups (1-100 pM in gp100). Notably, the 1-SNAs even produced detectable T-cell proliferation at 100 fM treatment while the two other SNAs failed to show any effect. The calculated EC₅₀ values indicated that the 1-SNA (EC₅₀=2.3 pM) was approximately three times more potent than the 2-SNA (EC₅₀=6.4 pM), which itself was approximately three times more efficacious than the 3-SNA (EC₅₀=18 pM). This observation revealed the significance of antigen conjugation chemistry on the ability of SNAs to induce antigen-specific T-cell proliferation.

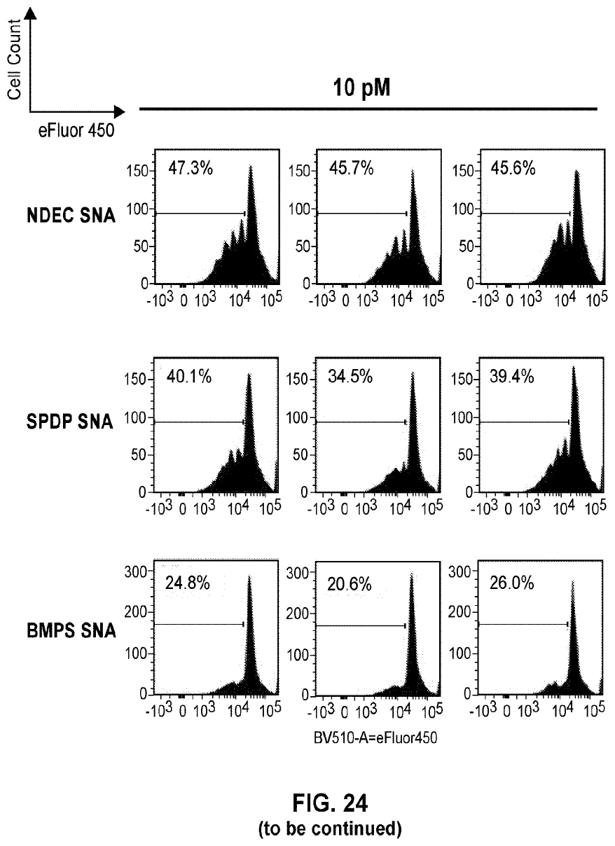

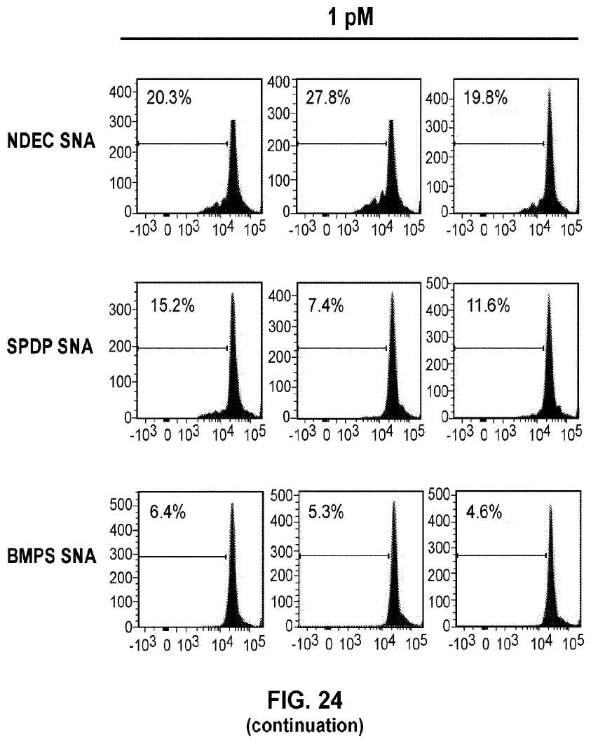

T-Cell Proliferation. Antigen specific T-cell proliferation was measured using genotyped pmel mice. Whole splenocytes were harvested from the mice, stained with eFluor 450 dye, and cultured under the different treatment conditions for 72 hours. Following treatment, the CD8 marker was stained and flow cytometry was run to measure the proliferation ratio of CD8⁺ T-cells. Gating stagnate was based on read-out from a medium only treatment group. All experiments were carried out in triplicate. See FIG. 24 for results.

To further evaluate the impact of conjugation chemistry on T-cell activation, the release of cytokines—IFN-γ, TNF-α, granzyme-B, and IL-6—were quantified for all three SNAs using ELISA (FIG. 23C). Consistent with results of T-cell proliferation, it was shown that T-cells treated with the traceless 1-SNAs secrete higher levels of the cytokine activation markers IFN-γ and IL-6, compared to the 2-SNA and 3-SNA groups at the 10 pM concentration. This showed that traceless NDEC conjugation chemistry leads to higher T-cell activation. Granzyme B and TNF-α secretion, which resulted from 1-SNA treatment, were also higher than all other groups at 10 pM treatment condition, indicating the increased potential of T-cell-mediated killing of tumor cells.

APC activation and SNA uptake. APC activation after treatment with SNAs was measured using bone marrow-derived DCs that were cultured and stimulated by GM-CSF for 6 days prior to treatment. Biotin-positive selection kit (Stem Cell Catalog #18556) was used to select DCs with the CD11c marker. Then, 3E5 cells were treated with three different SNAs at a 100 nM concentration in an incubator (37° C., 5% CO₂) for 24 hours before measuring the activation markers. Flow cytometry data shows that all SNAs achieved the indistinguishable APC activation via CD40, CD80 expression. Uptake was measured by comparing the amount of Cy5 fluorescence in DCs using flow cytometry. Cells treated with the three SNAs show indistinguishable levels of Cy5 median fluorescence.

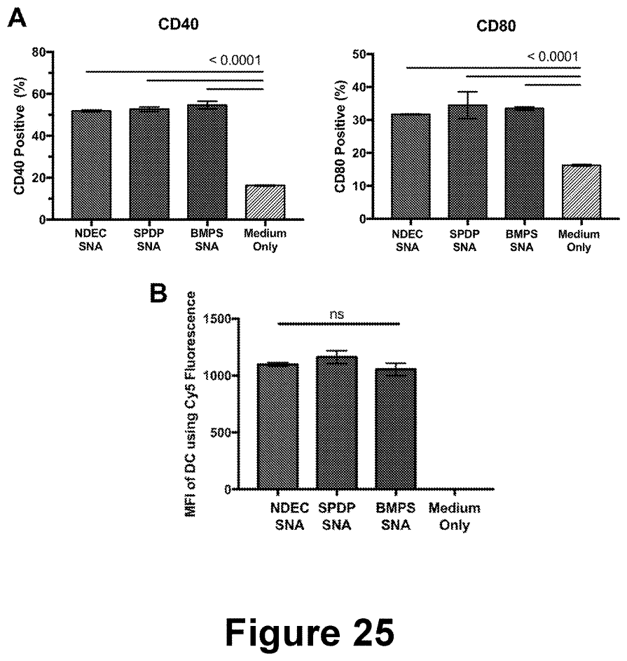

Optimum T-cell activation and proliferation depend on MHC-antigen-TCR binding as well as the activation state of the APCs. The observed differences in SNA efficacy could be due to different levels of APC activation. Therefore, the activation levels of DCs across the SNA types were compared by quantifying the expression of the costimulatory markers, CD40 and CD80 (FIG. 25). All SNA types caused upregulation in the expression of the two receptors compared to a medium only control. No difference in APC activation between the three SNA types was observed, indicating that the activation of DCs, caused by the interaction of CpG oligonucleotides with TLR receptors in the endosomes, is likely independent of the linkage chemistry used to form the gp100-DNA conjugates.

Taken together, these data showed that the choice of linker chemistry used to conjugate an antigen, gp100, to the immunostimulatory SNA had a significant impact on potency and has implications for vaccine development. Importantly, these findings showed that the chemistry used to conjugate the antigen to an SNA cannot be chosen based simply on synthetic convenience, but instead the choice should be made by considering its impact on the immunogenicity of the delivered antigen. This knowledge underscores the impact of conjugation chemistry on immunostimulatory nanotherapeutic constructs and inform the design of future vaccines, beyond those based upon the SNA architecture.

### Example 3

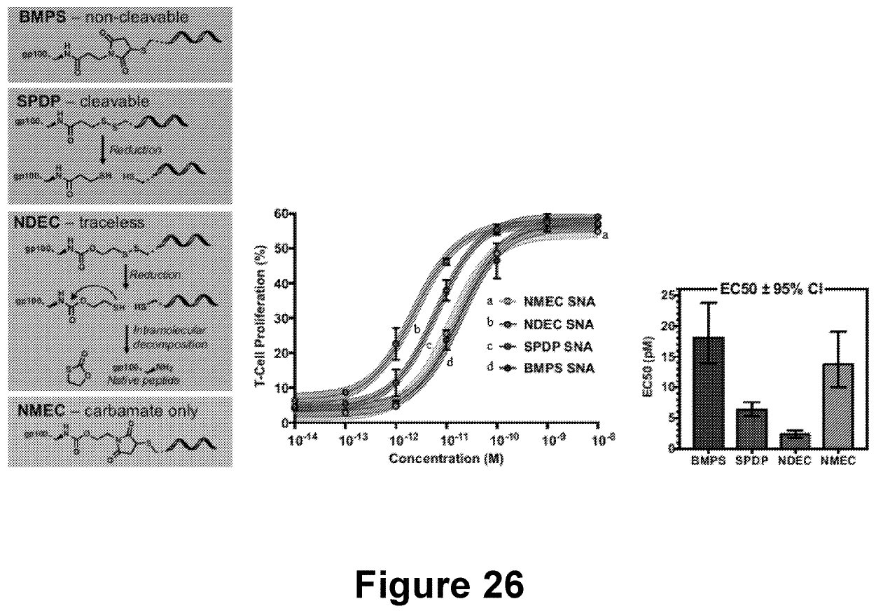

Additional experiments were performed to test the efficacy and kinetics of the various linkers disclosed herein. FIG. 26 shows results from experiments demonstrating that a carbamate linkage alone does not provide T-cell proliferation benefit. FIG. 26 shows the various linkers utilized (left panel), T-cell proliferation data for each linker (middle panel), and EC₅₀ data (right panel). NMEC SNAs were shown to possess an EC₅₀ of 1.8 pM. There was no change in efficacy compared to BMPS. The experiments also demonstrated that a disulfide was necessary for the functioning of the linker, but not sufficient.

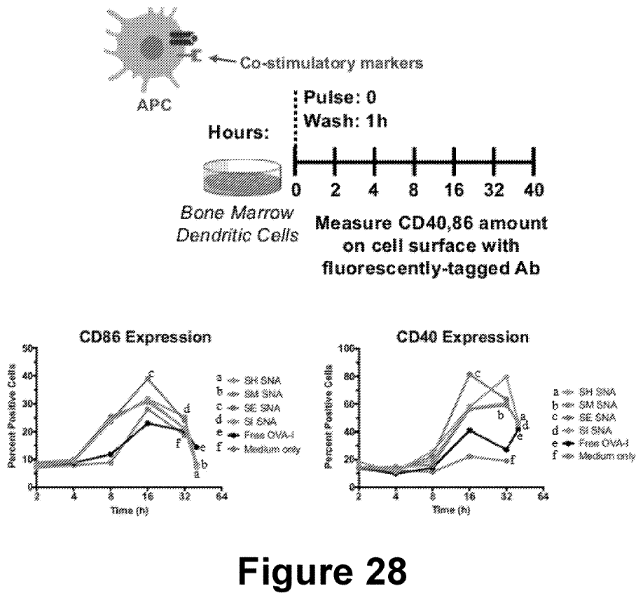

FIG. 28 shows that the dendritic cell (DC) surface markers CD40 and CD86 showed similar APC activation between the linkers depicted in FIG. 27. Costimulatory marker (CD86, CD40) expression varied over time between all SNA types; the data suggested that the kinetics of DC activation are similar between SNAs (FIG. 28).

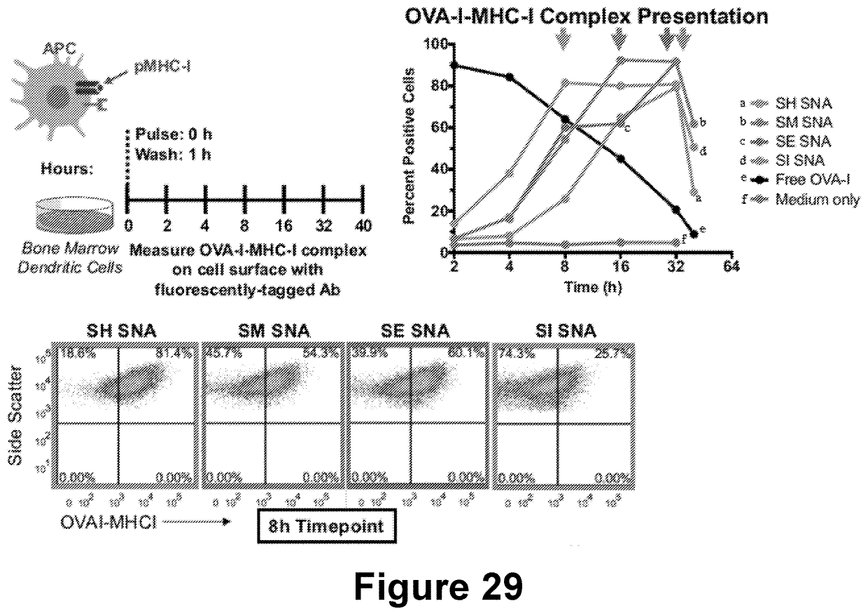

FIG. 29 depicts results of experiments showing that the presentation of OVA-I-MHC-I complex on the surface of dendritic cells varies between the linkers. The experiments showed that OVA-I presentation on surface MHC-I molecules varies over time between the SNA types. The experiments also showed that the linker type affects the kinetics of antigen presentation.

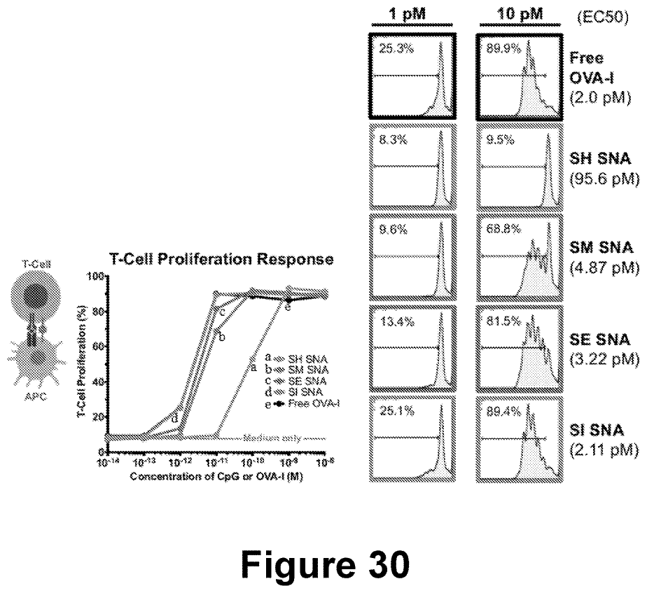

Further experiments showed that T-cell proliferation varied between the linker types. FIG. 30 depicts the results of the experiments, and shows that the linker design affects the efficacy of SNAs to elicit T-cell proliferation. No major differences were observed between the slower linkers.

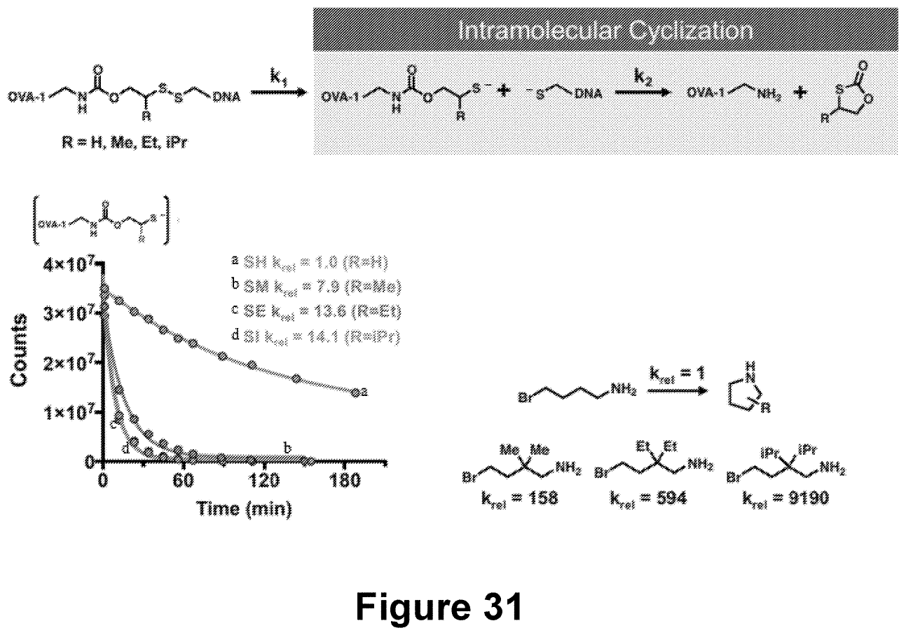

FIG. 31 shows that additional steric bulk increased the rate of cyclization. Without being bound by theory, this increase in rate is likely due to a Thorp-Ingold effect, which describes an increase in intramolecular reaction rates with increasingly bulky substituents, which is driven by a decrease in linear conformations that place the reactive groups far from each other. [Brown et al., J. Org. Chem. 21: 1046 (1956)] The experiments in FIG. 31 were performed using 100 mM phosphate buffer at pH 7.4, 5.0 um OVA1-DNA conjugate, and 19.3 mM TCEP in the reaction. LC-TOF was performed using a C18 RP column, water:ACN gradient with 0.1% formic acid.

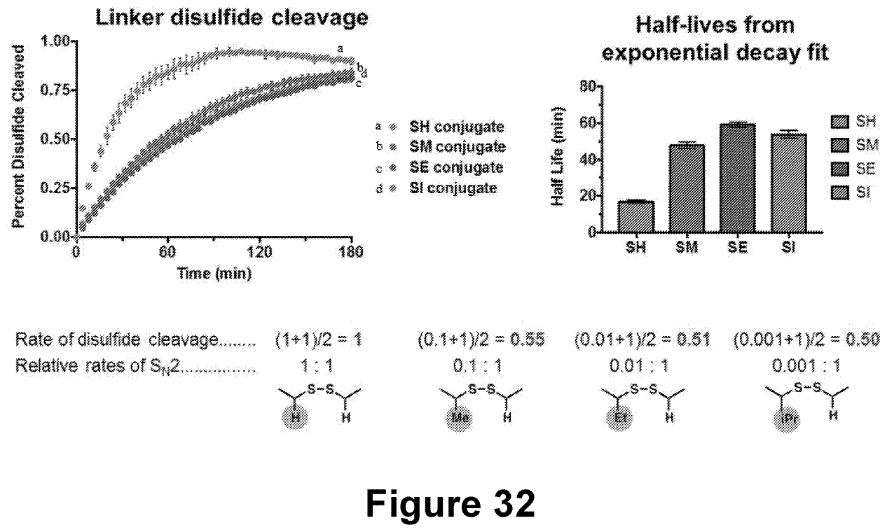

FIG. 32 shows results of experiments quantifying the rates of disulfide cleavage using the FITC-Eclipse quencher system. The reaction was performed in 1×PBS at pH 7.4 and 25° C. For the experiments, 76.2 nM conjugate and 14 mM glutathione were used (Ex 480, Em 520, scan every 4 minutes). The data showed that the original (SH) linker has a half-life of approximately 20 minutes, while the bulky linkers (SM, SE, and SI) all have similar half-lives of approximately one hour.

The experiments showed that steric bulk can be used to slow down the kinetics of disulfide cleavage, but it also increases the rate of cyclization. Dendritic cell (DC) activation did not differ between linker types, but OVA1-MHC presentation is affected by linker design. Finally, T-cell proliferation increased due to linker design.

### REFERENCES

(1) Zhang, G.; Yang, Z.; Lu, W.; Zhang, R.; Huang, Q.; Tian, M.; Li, L.; Liang, D.; Li, C. Biomaterials 2009, 30 (10), 1928-36.

(2) Liu, H.; Moynihan, K. D.; Zheng, Y.; Szeto, G. L.; Li, A. V.; Huang, B.; Van Egeren, D. S.; Park, C.; Irvine, D. J. Nature 2014, 507 (7493), 519-22.

(3) Townson, J. L.; Lin, Y. S.; Agola, J. O.; Carnes, E. C.; Leong, H. S.; Lewis, J. D.; Haynes, C. L.; Brinker, C. J. J. Am. Chem. Soc. 2013, 135 (43), 16030-3.

(4) Kim, H. K.; Thompson, D. H.; Jang, H. S.; Chung, Y. J.; Van den Bossche, J. ACS Appl. Mater. Interfaces 2013, 5 (12), 5648-58.

(5) Dunn, S. S.; Tian, S.; Blake, S.; Wang, J.; Galloway, A. L.; Murphy, A.; Pohlhaus, P. D.; Rolland, J. P.; Napier, M. E.; DeSimone, J. M. J. Am. Chem. Soc. 2012, 134 (17), 7423-30.

(6) Fang, R. H.; Hu, C. M.; Luk, B. T.; Gao, W.; Copp, J. A.; Tai, Y.; O'Connor, D. E.; Zhang, L. Nano Lett. 2014, 14 (4), 2181-8.

(7) Perrault, S. D.; Walkey, C.; Jennings, T.; Fischer, H. C.; Chan, W. C. Nano Lett. 2009, 9 (5), 1909-15.

(8) Li, J.; Wang, W.; He, Y.; Li, Y.; Yan, E. Z.; Zhang, K.; Irvine, D. J.; Hammond, P. T. ACS Nano 2017, 11 (3), 2531-2544.

(9) Dong, Y.; Dorkin, J. R.; Wang, W.; Chang, P. H.; Webber, M. J.; Tang, B. C.; Yang, J.; Abutbul-lonita, I.; Danino, D.; DeRosa, F.; Heartlein, M.; Langer, R.; Anderson, D. G. Nano Lett. 2016, 16 (2), 842-8.

(10) Liu, X.; Lin, P.; Perrett, I.; Lin, J.; Liao, Y. P.; Chang, C. H.; Jiang, J.; Wu, N.; Donahue, T.; Wainberg, Z.; Nel, A. E.; Meng, H. J. Clin. Invest. 2017, 127 (5), 2007-2018.

(11) Taton, T. A.; Mirkin, C. A.; Letsinger, R. L. Science 2000, 289 (5485), 1757-60.

(12) Rosi, N. L.; Giljohann, D. A.; Thaxton, C. S.; Lytton-Jean, A. K.; Han, M. S.; Mirkin, C. A. Science 2006, 312 (5776), 1027-30.

(13) Radovic-Moreno, A. F.; Chernyak, N.; Mader, C. C.; Nallagatla, S.; Kang, R. S.; Hao, L. L.; Walker, D. A.; Halo, T. L.; Merkel, T. J.; Rische, C. H.; Anantatmula, S.; Burkhart, M.; Mirkin, C. A.; Gryaznov, S. M. Proc. Natl. Acad. Sci. U. S. A. 2015, 112 (13), 3892-3897.

(14) Hirosue, S.; Kourtis, I. C.; van der Vlies, A. J.; Hubbell, J. A.; Swartz, M. A. Vaccine 2010, 28 (50), 7897-7906.

(15) Suma, T.; Cui, J. W.; Mullner, M.; Fu, S. W.; Tran, J.; Noi, K. F.; Ju, Y.; Caruso, F. J. Am. Chem. Soc. 2017, 139 (11), 4009-4018.

(16) Xu, J.; Wang, J. J.; Luft, J. C.; Tian, S. M.; Owens, G.; Pandya, A. A.; Bergund, P.; Pohlhaus, P.; Maynor, B. W.; Smith, J.; Hubby, B.; Napier, M. E.; DeSimone, J. M. J. Am. Chem. Soc. 2012, 134 (21), 8774-8777.

(17) Bakker, A. B.; Schreurs, M. W.; Tafazzul, G.; de Boer, A. J.; Kawakami, Y.; Adema, G. J.; Figdor, C. G. Int. J. Cancer 1995, 62 (1), 97-102.

(18) de Titta, A.; Ballester, M.; Julier, Z.; Nembrini, C.; Jeanbart, L.; van der Vlies, A. J.; Swartz, M. A.; Hubbell, J. A. Proc. Natl. Acad. Sci. U. S. A. 2013, 110 (49), 19902-7.

(19) Klinman, D. M.; Barnhart, K. M.; Conover, J. Vaccine 1999, 17 (1), 19-25.

(20) Chen, J. W.; Zhao, M. K.; Feng, F. D.; Sizovs, A.; Wang, J. J. Am. Chem. Soc. 2013, 135 (30), 10938-10941.

(21) Dunn, S. S.; Tian, S. M.; Blake, S.; Wang, J.; Galloway, A. L.; Murphy, A.; Pohlhaus, P. D.; Rolland, J. P.; Napier, M. E.; DeSimone, J. M. J. Am. Chem. Soc. 2012, 134 (17), 7423-7430.

(22) Dutta, K.; Hu, D.; Zhao, B.; Ribbe, A. E.; Zhuang, J. M.; Thayumanavan, S. J. Am. Chem. Soc. 2017, 139 (16), 5676-5679.

(23) Saito, G.; Swanson, J. A.; Lee, K. D. Adv. Drug Delivery Rev. 2003, 55 (2), 199-215.

(24) Goldberg, M. S. Cell 2015, 161 (2), 201-204.

(25) Steinman, R. M.; Cohn, Z. A. J. Exp. Med. 1973, 137 (5), 1142-62.

(26) Banchereau, J.; Steinman, R. M. Nature 1998, 392 (6673), 245-52.

(27) Dominguez, D.; Ye, C.; Geng, Z.; Chen, S.; Fan, J.; Qin, L.; Long, A.; Wang, L.; Zhang, Z.; Zhang, Y.; Fang, D.; Kuzel, T. M.; Zhang, B. J. Immunol. 2017, 198 (3), 1365-1375.

(28) Qin, L.; Dominguez, D.; Chen, S.; Fan, J.; Long, A.; Zhang, M.; Fang, D.; Zhang, Y.; Kuzel, T. M.; Zhang, B. Oncotarget 2016, 7 (38), 61069-61080.

What is claimed is:

1. A spherical nucleic acid (SNA) comprising a nanoparticle and a double stranded oligonucleotide, wherein:
a first strand of the double stranded oligonucleotide comprises an associative moiety that allows association of the double-stranded oligonucleotide with the nanoparticle;
a second strand of the double stranded oligonucleotide comprises an antigen that is attached to the second strand through a linker;
wherein the first strand and the second strand comprise sequences that are sufficiently complementary to each other to hybridize to form the double stranded oligonucleotide.

2. The SNA of claim 1, wherein the first strand comprises an immunomodulatory nucleotide sequence.

3. The SNA of any one of claim 1-3, wherein the first strand comprises a sequence that is a toll-like receptor (TLR) agonist.

4. The SNA of claim 4, wherein the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLR5), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13).

5. The SNA of any one of claims 2-4, wherein the first strand comprises a CpG nucleotide sequence.

6. The SNA of any one of claims 1-5, wherein the second strand comprises a carbamate alkylene dithiolate linker.

7. The SNA of claim 6, wherein the second strand comprises Antigen-NH—C(O)—O—C₂₋₅alkylene-S—S—C₂₋₇alkylene-Oligonucleotide, or Antigen-NH—C(O)—O—CH₂—Ar—S—S—C₂₋₇alkylene-Oligonucleotide, and Ar comprises a meta- or para-substituted phenyl.

8. The SNA of claim 7, wherein the second strand comprises Antigen-NH—C(O)—O—C₂₋₄alkylene-CH(X)—S—S—CH(Y)C₂₋₆alkylene-Oligonucleotide, and X and Y are each independently H, Me, Et, or iPr.

9. The SNA of claim 7, wherein the second strand comprises Antigen-NH—C(O)—O—CH₂—Ar—S—S—CHXC₂₋₆alkylene-Oligonucleotide, and X is Me, Et, or iPr.

10. The SNA of any one of claims 1-5, wherein the second strand comprises an amide alkylene dithiolate linker.

11. The SNA of claim 10, wherein the second strand comprise Antigen-NH—C(O)—C₂₋₅alkylene-S—S—C₂₋₇alkylene-Oligonucleotide.

12. The SNA of claim 11, wherein the second strand comprises Antigen-NH—CO)—CH(X)C₂₋₄alkylene-S—S—CH(Y)C₂₋₆alkylene-Oligonucleotide, and X and Y are each independently H, Me, Et, or iPr.

13. The SNA of any one of claims 1-5, wherein the second strand comprises a amide alkylene thio-succinimidyl linker.

14. The SNA of claim 13, wherein the second strand comprises Antigen-NH—C(O)—C₂₋₄alkylene-N-succinimidyl-S—C₂₋₆alkylene-Oligonucleotide.

15. The SNA of any one of claims 1-14, wherein the antigen is a tumor associated antigen, a tumor specific antigen, a neo-antigen.

16. The SNA of claim 15, wherein the antigen is OVA1, MSLN, P53, Ras, a melanoma related antigen, a HPV related antigen, a prostate cancer related antigen, an ovarian cancer related antigen, a breast cancer related antigen, a hepatocellular carcinoma related antigen, a bowel cancer related antigen, or human papillomavirus (HPV) E7 nuclear protein.

17. The SNA of any one of claims 1-16, wherein the nanoparticle is a liposome.

18. The SNA of claim 17, wherein the liposome comprises a lipid selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), and cholesterol.

19. The SNA of any one of claims 1-18, wherein the associative moiety is tocopherol, cholesterol, 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-di-(9Z-octadecenoyI)-sn-glycero-3-phosphoethanolamine (DOPE), or 1,2-dihexadecanoyl-sn-glycero-3-phosphoethanolamine (DPPE).

20. The SNA of any one of claims 1-19, wherein the double stranded oligonucleotide comprises RNA or DNA.

21. The SNA of any one of claims 1-20, further comprising an additional oligonucleotide.

22. The SNA of claim 21, wherein the additional oligonucleotide comprises RNA or DNA.

23. The SNA of claim 22, wherein said RNA is a non-coding RNA.

24. The SNA of claim 23, wherein said non-coding RNA is an inhibitory RNA (RNAi).

25. The SNA of claim 23 or claim 24, wherein the RNAi is selected from the group consisting of a small inhibitory RNA (siRNA), a single-stranded RNA (ssRNA) that forms a triplex with double stranded DNA, and a ribozyme.

26. The SNA of claim 23 or claim 24, wherein the RNA is a microRNA.

27. The SNA of claim 22, wherein said DNA is antisense-DNA.

28. The SNA of any one of claims 1-27, wherein the nanoparticle has a diameter of 50 nanometers or less.

29. The SNA of any one of claims 1-28 comprising about 10 to about 80 double stranded oligonucleotides.

30. The SNA of claim 29 comprising 75 double stranded oligonucleotides.

31. A composition comprising the SNA of any one of claims 1-30 in a pharmaceutically acceptable carrier.

32. The composition of claim 31, wherein the composition is capable of generating an immune response in an individual upon administration to the individual.

33. The composition of claim 32, wherein immune response comprises antibody generation or a protective immune response.

34. A vaccine comprising the composition of any one of claims 31-33, and an adjuvant.

35. The composition of claim 32, wherein the immune response is a neutralizing antibody response or a protective antibody response.

36. A method of producing an immune response to cancer in an individual, comprising administering to the individual an effective amount of the composition of claims 31-33, or the vaccine of claim 34, thereby producing an immune response to cancer in the individual.

37. A method of inhibiting expression of a gene comprising hybridizing a polynucleotide encoding the gene with one or more oligonucleotides complementary to all or a portion of the polynucleotide, the oligonucleotide being the additional oligonucleotide of the SNA of any one of claims 21-30, wherein hybridizing between the polynucleotide and the oligonucleotide occurs over a length of the polynucleotide with a degree of complementarity sufficient to inhibit expression of the gene product.

38. The method of claim 37 wherein expression of the gene product is inhibited in vivo.

39. The method of claim 37 wherein expression of the gene product is inhibited in vitro.

40. A method for up-regulating activity of a toll-like receptor (TLR) comprising contacting a cell having the TLR with a SNA of any one of claims 1-30.

41. The method of claim 40 wherein the double stranded oligonucleotide comprises a TLR agonist.

42. The method of claim 40 or claim 41 wherein the TLR is chosen from the group consisting of toll-like receptor 1 (TLR1), toll-like receptor 2 (TLR2), toll-like receptor 3 (TLR3), toll-like receptor 4 (TLR4), toll-like receptor 5 (TLRS), toll-like receptor 6 (TLR6), toll-like receptor 7 (TLR7), toll-like receptor 8 (TLR8), toll-like receptor 9 (TLR9), toll-like receptor 10 (TLR10), toll-like receptor 11 (TLR11), toll-like receptor 12 (TLR12), and toll-like receptor 13 (TLR13).

43. The method of any one of claims 40-42 which is performed in vitro.

44. The method of any one of claims 40-42 which is performed in vivo.

45. The method of any one of claims 40-44, wherein the cell is an antigen presenting cell (APC).

46. The method of claim 45, wherein the APC is a dendritic cell.

47. The method of claim 45, wherein the cell is a leukocyte.

48. The method of claim 47, wherein the leukocyte is a phagocyte, an innate lymphoid cell, a mast cell, an eosinophil, a basophil, a natural killer (NK) cell, a T cell, or a B cell.

49. The method of claim 48, wherein the phagocyte is a macrophage, a neutrophil, or a dendritic cell.

50. A method of immunizing an individual against cancer comprising administering to the individual an effective amount of the composition of any one of claims 31-33, thereby immunizing the individual against cancer.

51. The method of claim 50, wherein the composition is a cancer vaccine.

52. The method of claim 50 or 51, wherein the cancer is selected from the group consisting of bladder cancer, breast cancer, colon and rectal cancer, endometrial cancer, glioblastoma, kidney cancer, leukemia, liver cancer, lung cancer, melanoma, non-hodgkin lymphoma, osteocarcinoma, ovarian cancer, pancreatic cancer, prostate cancer, thyroid cancer, and human papilloma virus-induced cancer.


SEQUENCE LISTING (4 sequences)

SEQ ID NO: 1 - protein, 9 aa, Homo sapiens
KVPRNQDWL

SEQ ID NO: 2 - DNA, 20 bp, Artificial Sequence
  Synthetic Polynucleotide
  misc_feature: CpG Anchor (PS)
  misc_feature (20)..(20): (Cy5) (Sp18)2 Cholesterol
tccatgacgttcctgacgtt

SEQ ID NO: 3 - DNA, 20 bp, Artificial Sequence
  Synthetic Polynucleotide
  misc_feature: Conjugate (CpG complement, PO)
  misc_feature (20)..(20): Sp18 C3 Thiol
aacgtcaggaacgtcatgga

SEQ ID NO: 4 - DNA, 20 bp, Artificial Sequence
  Synthetic Polynucleotide
  misc_feature: FRET conjugate (PO)
  misc_feature (20)..(20): Sp18 Eclipse Quencher C3 Thiol
aacgtcaggaacgtcatgga